SEQUENCE LISTING (15 sequences)

SEQ ID NO: 1 - DNA, 1452 bp, Methanosarcina acetivorans
  source 1..1452: mol_type = genomic DNA
atgagagatggtgaacagactccgggagttgctttaacaagggaaaagaagctactcatcgcgcgtgcattagatgagatgagaattaatgtcatcgaagccgggtctgctattaccagtgccggagaaagagagtccattaaggcagttgctaatgctggattagacgcagaaatctgtagttattgtagaattgtgaagatggatgtggatcatgccctcgagtgtgatgttgattcaattcatttggtagctccagtgagtgacctccacattaaaaccaagatcaagaaggatagagatactgttagacagatcgccgcagaggtcacagagtacgcaaaggatcatggtttaatcgttgaactatccggcgaggacgcctcgagagccgatccagaatttttaaaggcaatttactctgacggtattgacgcgggagctgacagattgtgcttttgcgataccgtcggtctattggttccagagaaaacaactgagatctttcgtgacctttccagttccttgaaggcacctatttctattcattgtcacaacgacttcggccttgctacagccaacacagtcgctgcattagctgctggtgcaaagcagtcccatgtgacaattaacggacttggtgaaagagctggtaatgcctcgttggaagaagttgtcatgtcgctcgagtggttatacaagtacgacactggaatcaaacatgagcagatctatagaacatcaagattggtttcgagattaacaggtattcccgtgagtccaaataaggcattggttggtggtaatgctttcactcacgaagcaggaatccatgtccacggcttgttagcggataagtcgacctatgaacctatgtcgccagagtacatcggtagacaaagacaaatcgtgcttggcaagcacgcgggtcgttcttctattactttggcattgaaggaaatgggtttggaggccgatgaagctcaaactgaagaaatctttaacagagttaagcaaatgggtgaccagggtaagcatattactgatgcagatttgcaaaccattgctgaaacagtcttagacatttataaggagcctatcgtgaaattagaagaatttacaattgtctccggaaatcgtgtcacccctaccgcctctattaaattgaatgtgaaagacaaggagatcgtccaggccggtatcggtaatggtcctgttgatgcagtgattaacgctattcgtagagccgtgagttcttgcgccgaggatgttgttcttgaagaataccatgttgattccataactggtggtacggatgcacttgttgaagtgagagtgaagctatcaaaaaacggtaaggttatcacagcttcaggtgctagaactgatataatcatggcctcagttgaagcagtcatgaatggtatgaacaggttaattcgtgaagaataa

SEQ ID NO: 2 - DNA, 1470 bp, Archaeoglobus fulgidus
  source 1..1470: mol_type = genomic DNA
atgcaagtcaagatccttgatacaacattgcgtgacggtgagcaaacccctggtgtttctttgtccgtcgaacaaaaggtgatgatcgcagaagctctcgacaaccttggagttgatattatcgaagcgggtactgccatagcctctgaaggggattttcaagcaattaaggaaatttcacaaagaggtctcaatgccgaaatctgttcattcgcgagaattaagcgagaggacatcgacgctgccgccgatgctggtgcagagtcaatctttatggtggctccatcgtctgatattcatataaacgccaagtttccaggaaaggacagagactacgtcatcgagaaatcagtcgaagctattgaatatgcaaaggaaagaggcctcattgtggaattcggtgctgaagatgcatcaagagccgacctcgatttcgttattcaattgttcaaaagagcggaggaggcaaaggccgatagaatcacattcgcggacactgttggagtgctttctcctgaaaagatggaagaaattgtgagaaagatcaaagcaaaggttaaattgccattagctatacactgtcatgacgatttcggcttggcaaccgctaacactatttttggtattaaggccggcgcggaagaatttcatggcacgattaacggtttgggtgagagggcaggcaatgccgccatcgaagaggttgttatcgcattggaatacctttacggtattaagaccaaaattaagaaggaaagattgtacaatacttctaagctcgtggagaagttgtcccgtgtcgtcgttccaccaaacaagccaattgtcggagataacgctttcactcatgagtccggtatccatacttctgcattgttcagagatgcaaaatcctacgagcccatctcgcctgaagttgttggtaggaagagggtcatcgttttgggtaagcacgctggtagggcaagcgttgaagcaattatgaatgaattaggttacaaggctaccccggaacagatgaaggaaattctagctagaattaaggaaattggtgataagggtaaaagagttaccgatgctgatgttcgaacaataattgaaactgtgttgcaaattaaaagagaaaaaaaagtcaagcttgaggatttagcaatcttctctggtaagaacgtcatgcccatggcgtcagtcaagttgaaaattgacggtcaagagagaattgaggccgctgttggattaggaccagtcgatgccgcaattaacgcaatcaggagagcaattaaagaatttgcggatatcaaattagtttcctaccatgttgacgccattacaggaggtacggacgccctcgttgatgtcgttgttcagttgaagaaagacaacaagattgttacggcacgtggtgcgaggacagatattattatggcatccgttgaagcattcatcgagggtattaatatgctcttctaa

SEQ ID NO: 3 - DNA, 1476 bp, Methanocaldococcus jannaschii
  source 1..1476: mol_type = genomic DNA
atgatggttaggattttcgacactactttaagagatggtgaacagactccaggtgtttctctgaccccaaacgataagttggaaatcgcaaagaaattggatgaattgggggttgatgttattgaggcaggatcagctattacttctaagggagaaagagaaggtatcaagctcattactaaggagggtttgaacgcagaaatctgttccttcgtccgtgcattgcctgtcgatatcgatgcggcgttagagtgtgatgtcgattcggttcatttagtggtgcctacatccccaatccatatgaagtacaaattgaggaagacggaggatgaagtcttggaaacggcgttgaaggcagtcgaatatgctaaggagcatggtttaatagtcgagttgtcggcggaagatgcgaccagatccgatgttaacttcttgatcaagttgttcaatgaaggtgaaaaggtcggtgcagatagagtctgtgtttgtgataccgttggtgtccttacacctcaaaagtcacaagaattgtttaagaagattactgaaaatgttaacctccccgtttcagtccattgccataacgattttggtatggcgacagctaatacatgttctgcggtcttgggtggcgctgtccagtgtcatgtcacagttaacggaattggtgaaagagcaggaaatgcctcattggaggaagttgttgccgcattaaaaattttgtacggttatgatactaagattaaaatggaaaagttgtacgaagtgtcccgtatcgtttcgagattaatgaagttgcctgtgcctccaaacaaagccatcgtgggcgataatgctttcgcccatgaagccggtattcatgttgatggtctcattaaaaacactgaaacgtatgagcctatcaagccagagatggttggtaaccgtcgtagaattattttgggtaaacattctggtagaaaagcactcaaatataagttagacttgatgggaattaacgtttctgatgaacagttgaataagatttatgagcgtgtcaaggagttcggtgacttgggaaagtatatttccgatgcagatttactggcaattgtgagagaagttacgggtaagttagttgaggaaaagattaagttggacgagttgaccgttgtttcggggaataagattactccaattgcctccgttaagctccattacaagggagaggatattactttgattgaaacagcctatggcgttggaccagtggacgcggcgatcaatgcagtcagaaaggcaatttcgggtgttgctgacatcaagttagttgagtacagggttgaggccatcgggggtggtactgatgcattgatcgaagtcgttgtcaagcttagaaagggcactgaaatagtcgaagtgaggaagtccgatgccgacatcattagagcttcagtcgacgcagtgatggaaggtatcaacatgttacttaattag

SEQ ID NO: 4 - DNA, 1512 bp, Methanoculleus marisnigri
  source 1..1512: mol_type = genomic DNA
atgattgtcttgtttgttgaacccattaggttctttgataccacattaagagatggtgagcaaactccaggcgttagtttgacacctgccggaaagctggaaattgcaacacacttagccgatgttggggtccatgttatcgaagcaggttccgcagcggcttctgttggagaacgtgagtccattagagcgattgcagacgcaggtttagcagccgagtgttgtacctacgtcagggcattaccaggcgatattgatttagctgccgatgcgggcgccgattctgtccacttggtcgttcctgtctctgatttgcacattgctaagaagttgaggaagactagagaacaggtttctgagatggcctggtccgcagttgaatatgccaaggaaagaggtttggttgttgagttgtcaggtgaagatgcgtcgagagcagatcaggattttttggcagaagtttttagagaaggcgttgaaagaggtgctgatcgattatgtttttgtgataccgtcggtttactgacaccagaaagagccgccgcaattattccacctcttcttttcgcgcctttatcgatccactgtcatgatgatttggggtttggtttagcaaccacagtcgcagccttgagggctggtgctacttgcgcacatgttacagttaatggtttaggcgaacgtgcaggtaacacttcgttagaagaattggtcatggcattggaagttctttatggcgtcgatacgggtattgccactgaagaattgtatccattaagtactcacgtcgcaagactcacaggtgtcccattggctaccaataagcctattgttggcgaaatggcgttcactcatgagtcaggaatccacgctcatggtgttatgcgggacgcatccacgtatgaacccttgcaacctgagagagtaggtagaagaagaagaatcgttttaggtaagcactctggttcagccgccgttgaagctgctttgcatgatatgggttatgcaccatcggccgctcaactcaaggaaattgtcgatagaattaaaagacttggtgatgcaggtatgagaatcaccgacgcagatattatggcaattgctgatacagtcatggaaatcgaatttacaccgtgtatcgaactgaggcaattcacaatcgtttcaggatctaacgcaatcccaactgcttcggtcaccatgctagtgagaggtgaagaaatcacgggtgcagccgtcggtacaggtccagttgacgcagcaattagagctttacaaagatccgttgctgatgttggttctgtcagattagatgagtactcggttgatgccatcaccggtggtacagatgccttggtggatgtctccgttaagttatctaaagacggtaagaccgttactagtagaggtgccagaactgacattatcatggcatctgttgaagctgttattgcaggtatgaacaggcttctcagagaagaacacgaagatagatcgcaagattccgattaa

SEQ ID NO: 5 - DNA, 1530 bp, Methanopyrus kandleri
  source 1..1530: mol_type = genomic DNA
atgagagaagctaacgcagatgcagacccaccagatgaggttcggatctttgatactactttacgtgatggcgaacaaactcctggcgttgcgttaacacctgaagaaaaacttagaatcgctagaaaattggatgaaattggtgttgataccatagaagcaggtttcgcagcggcttcggaaggagaattgaaagcaattagaagaatcgcaagagaagaattggacgcggaggtttgttcgatggcgagaatggtcaagggagatgttgatgcagccgtggaggcggaagccgatgccgtccacatagtcgttccaacttcagaggttcatgttaagaagaagttaagaatggatagggaagaggtcttggaaagagccagagaggtcgttgaatatgctagagatcatggtttgaccgttgaaatttcaactgaagatggtactagaacagaattagaatatttgtatgaggtgtttgatgcatgcttagaggctggagctgaaaggttgggttacaacgataccgtcggtgtcatggcacctgaaggtatgttcttggcagtcaagaaattacgtgagagagtcggtgaagacgttatcctctcagttcactgtcacgatgactttggtatggcaactgctaatacggtggcagcagttagggcaggtgctagacaagttcatgttacagttaatggtattggtgaaagagctggtaacgcggcattagaagaagttgtcgtcgttttggaagagttatacggtgtggatactggaatccgtactgaaagattgaccgagctctctaaattggtcgaaagattgactggcgtcagagttcccccaaacaaggccgtcgttggtgaaaacgcctttacacacgaatccggtattcatgccgatggtattttaaaggatgaatctacatacgaacccatccctcccgagaaggtcggtcatgaaagacgtttcgtcttgggtaagcatgttggtacctcagtcattaggaagaagttgaagcagatgggggttgacgtcgacgatgaacagttgcttgaaatcttgcgtcgtcttaaaagattgggtgatcgtggtaaaagaattacagaggcagatctcagagctattgcagaggatgttctaggtagaccagcagagagagacatcgaagttgaagatttcacaacagtgactggaaagcgtacaattccaactgcgtcgattgttgtcaaaattgatggtacacgtaaagaagctgcttcaaccggtgtcggtccagtcgatgcaactatcaaggctttagagcgtgcattgaaggatcagggtattgatttcgaactggttgagtatcgagcagaagcattgactggaggtaccgatgccattacgcatgttgacgtcaagttgagggatcctgaaactggtgatattgtccactcaggttcgtcgagagaagatattgtcgttgctagtcttgaggccttcattgatggtattaactctttgatggcaagaaagagatcttga

SEQ ID NO: 6 - DNA, 1545 bp, Methanococcus maripaludis
  source 1..1545: mol_type = genomic DNA
atggaatcctacttgcactctaacgagatcatcaagaattccttaaagtctatgaaattgcccaaaaaggttagagttttcgatactacactccgggacggtgaacaaactcctggtgtctccttgaccccagatcagaagctagacattgccaccaagttgtcagaaattggtgttgatgcaattgaggcaggtttccctgtttccagcgagggtgaacaagaatcgatcaagaagattacctctatgggtttgaacgctgaaatttgcggtcttgctagagctgtgaagaaggatatcgatattgccatcgactgtggtgtcgattccattcatacttttattgcaacttctcctttacatagggagtataagctcaagatgtctaaggagaagatcattgatatcgcaattgaatccattgaatacatcaaggagcacggtatcattgtcgaattctcagcggaagacgcgactcgtacagaattagattatttaaaggaggtttataagaaggccgtcgaagctggagctgacagaatcaacgtccccgatactgtcggtgttatggtgccccattcgatgacgtatttgatctcagaattgaagaaggacatcaaagttccattaagtgtccactgtcataacgactttggtattgcagtttctaattcggttgccgcagttgaagcaggtgctgaacaggtccactgtacagtcaacggattgggtgagagagcaggcaatgcatctctcgaggaaaccgtgatgactttgaatatggtctatggtattgagacaaacgttgataccaagatgctaactaagctatctagaatcgtgtctaactacacaggtattaagacacagccaaacaaagcaattgtcggtgagaattctttcgcacacgaatctggtattcatgcgcacggcgttttggcccacgcattgacctacgagccgatcgatcctgcaatcgtgggaaacaaaagacgtatcgtcttgggtaagcactccggtgcccacgcgattaaatctaaattgtctgagatcggtgttgaaatcggtgacgctctatcgaaggaacaattctgtgaaattgttgagagagttaaggcaattggtgataagggtaagttggttaccgatgcagatgtcatggcaataacagaggatatcactcaacgaacaatcaagagtgaaagaattgtcgatttagaacaattcgcagtcatgacaggaaataacgttctcccaactgctagtgttgcgttgaaggtcagagacaagatttataaaacttccgagttgggtgtcggaccagttgacgctgcacttaaggcaatccaggcggcggtgggagaaaacatcaggctcaatgagtacaatatttcagcgatctcaggtggtactgatgccattgcagaagtgacagttagattggagaaccatgagaaggaagtcattgcaaaggctactggtgacgacgtcgtgaaggcttctgttgaagcggtcatagatggtattaataaactcatgtcctag

SEQ ID NO: 7 - DNA, 1587 bp, Geobacter sulfurreducens
  source 1..1587: mol_type = genomic DNA
atgtcgttagtcaagctttatgatacgactttgagagacggaacacaggcagaggatatctccttcttggttgaagataagatcagaattgctcataaactcgatgagattggaatccactacatcgaagggggttggccaggctcgaaccccaaggacgtcgcatttttcaaggatattaagaaagaaaagttgtcccaagcaaagatcgcggcgtttggttccactagaagagccaaggttacacccgataaggaccataacctcaaaactctcattcaagccgagccagacgtctgtactattttcggcaagacatgggacttccacgtccatgaagcattgagaatttccttggaagaaaacttggagctgatttttgattcgttggaatatttgaaggcgaacgtaccagaagtcttctacgatgcagaacatttttttgacggttataaagcaaatccagactatgcgattaagacattgaaggcagctcaggatgctaaggctgattgtattgtcttgtgtgatactaacggcggtaccatgccattcgaattagttgaaattattagagaggttagaaagcatataacagctccattgggtattcatactcataacgactccgaatgcgcagtcgctaactctttacatgcggtttcagaaggcattgtgcaagttcaaggaaccatcaacgggtttggggaaagatgtggtaacgcaaacttgtgttctattattcctgcgttgaaacttaaaatgaaaagagaatgtatcggtgatgatcaattgagaaaattgagagacttatctcgtttcgtttacgagttagccaatttgagcccaaacaaacatcaagcatacgtgggtaattccgctttcgcccacaaaggtggtgttcacgttagtgcaatccaacgtcatccagaaacttatgagcacttgagacctgagttagttggcaatatgaccagagttttggtttctgacctatcaggtagaagtaacattttggcaaaagcggaagagtttaacatcaaaatggattcaaaggacccagtcacattggaaatcttggaaaatattaaggaaatggagaacaggggttaccaattcgaaggtgctgaggcgtcgttcgaactgctgatgaaaagagcattgggtactcatcgtaagtttttttccgtcattggttttagagttatcgacgaaaagagacacgaggatcaaaagccattgtcggaagcaaccatcatggttaaagttggtggtaaaattgaacacacagcagctgaaggtaatggtcctgtcaatgccctcgataatgctttaagaaaggcattagagaagttctatccaagattgaaggaagttaaactcttggattataaggttcgtgtcttaccagcaggtcaaggtaccgccagttctattcgtgtcctaatcgaatcgggtgataaggaatcccgttggggtacggttggtgtttctgaaaacatagtggacgcatcttaccaagcccttctcgactccgttgaatataagctacataagtcggaagaaatcgaaggttctaaaaagtag

SEQ ID NO: 8 - DNA, 1605 bp, Streptomyces coelicolor
  source 1..1605: mol_type = genomic DNA
atgactgcaacgtcagaactcgacgactcatttcatgttttcgataccactttgagagatggtgcgcagcgtgaaggtattaacttaaccgttgccgacaagctcgctatcgcgagacaccttgatgattttggtgttggatttattgaaggaggctggccaggtgcaaatcctagggacacagaattttttgcaagagcaagacaagaaatcgatttcaaacatgctcaattggttgccttcggctctacacgtagagcaggtgcaaatgccgcagaagatcatcaagtcaaggcattattggattcgggtgcacaggtgattacattggtcgctaaatcacacgatagacacgttgaattagccctaagaactaccttggatgaaaatcttgcgatggtcgctgacaccgtttcccatctaaaggcccaaggtagaagagttttcgtggattgtgaacatttcttcgatggatatagggcgaaccctgaatacgctaagtccgttgttagaaccgcctccgaggcaggtgctgatgttgttgttctgtgtgatactaatggcggcatgttgccagcccaaattcaggccgttgttgcaactgttctagctgatacgggtgccagactgggtattcatgcgcaggatgataccggttgtgccgtcgcaaacacattagccgctgtggatgctggtgctactcacgttcagtgtactgctaatggttacggtgagagagtcggtaacgcaaatttattcccagtcgtcgcggctctagagctaaaatacgggaagcaggtgttgccagaaggcaggttacgggaaatgacgagaatttctcatgccatcgctgaggtcgtcaacttgactccatctactcaccaaccatacgttggtgtgtctgcctttgcacacaaagcgggtctgcacgcttcagcgatcaaggtggatccagatctttaccagcacattgaccctgaattagttggtaacactatgcgtatgttagtgtctgatatggcgggtagagcgtcaatcgaacttaagggtaaggaattgggcatcgatttgggtggagacagagagcttgtaggtagagttgttgaaagagttaaggagcgtgagttagcaggatacacctacgaagcagcggatgcatcctttgaattattattgcgtgctgaagccgagggtagaccactaaagtacttcgaagttgaatcctggagagctatcaccgaggacagaccagatggctctcatgcaaacgaagctactgtcaagttgtgggcaaagggtgaaagaattgttgcaactgctgaaggtaacggtcctgttaatgcactagatcgttccttaagagtcgcattggaaaagatttatcctgaattggcaaaattggatttggtcgattacaaggtcaggattttagagggagtgcatggtacacaaagtacaacaagggttctaatttcaacttccgacgggaccggagaatgggcaactgtgggtgttgctgaaaatgtcattgctgcatcgtggcaggccttagaggatgcatacacctatggtttattgcgtgcaggtgtcgcaccagcggagtag

SEQ ID NO: 9 - DNA, 1614 bp, Arabidopsis thaliana
  source 1..1614: mol_type = genomic DNA
atgagtacctcaatctctatttttgacacaaccttgagagatggtactcaaggtgagggtatctctcttacggcagaagataagattaaaattgcattgaagttggacgcactcggcgttcattatattgaaggaggtaaccctggttccaactccaaggatatcgaattctttagaagagcacgtgaattgaatttaagagcgaagcttactgctttcggttctacccgtcgtaagaattctttatgcgagcaagacgttaacttgttaaacttagtctcttcaggtgctaaggcggcaactctggtcggtaagacgtgggacttccacgttcacacagctttacaaactactttagaggaaaatttggcaatgatctacgatagcttagcgtatttgaagcaacacggtttagaagctattttcgactccgagcatttctttgatggcttcaaggctaaccctgattatgctatagcagccttgagaaaggctcaagaagctggtgcggactggattgttttgtgtgatacaaatggtggaacccttccaaacgaaattcaagatatcgttaagcaagtcaggaattcgatccaagcgccaatcggaatccacactcacaatgactgtgagcttgcggtggctaatactttggccgcagttaccgcgggtgcacgtcagattcagggaactattaatggttacggtgaaagatgtggcaacgccaatctatgctcgattttaccaaccttacagttgaagatgggttatcaagttgtcactccagagcaattaggttctttgacatccgttgcaagatacgttggtgaaatcgccaatgtggttttacctgtgaaccaaccttatgttggtaccgcagcttttgctcacaaaggtggtattcatgttagtgcgattttgaaggattccaagacatacgaacatatctctccagatctggtcggtaataaacaacgtgttttggtttcagaattagccggtcaatcgaatattttgtctaaagcacaggagatgggtttagctgtttcgaacgataacgctaacagtagagaagttattgagaagattaagaacctggagcaccagggatatcaattcgaaggtgcagatgcatctttggaattattgttgcgtgacgcctatggtgatgcagttgagatttttactgttgaaagctttaagatcttgatggaaaagtccccatctggtaatttaacagaagctattgtcaaattgaacgtttctggacaacaagtctacacagtcgctgagggtaatggtccagtgaacgctttagataatgctttgagaaaggctttgacccccttttatccagatatcaacggtatccatttgtccgattacaaggttcgtgttttagacgaaaaagatacaacggccgcgaaggttagggttctaatcgaatctactaactttaaggaatcttggtctaccgtcggcgtttcatctaacgttatcgaagcctcctgggaagcacttattgattctattcgttacgccttactaggcatgacgcaaacatccttttccccagaaagccctagagaaagtttgggtttggttaaccattaa

SEQ ID NO: 10 - DNA, 1698 bp, Chondrus crispus
  source 1..1698: mol_type = genomic DNA
atggataatactgagacaattacaggtgtcgttaagcttcctgatagaacaccaacgaaacacgacattgccactggtagagatccagatagagtgaagattttcgacaccacattgagggacggtgaacaatcaccaggagcatcgttgacagcggacgaaaagatggttatcgcaagacaactcgctaagttgggagtggacgttattgaggctgggttcccaatcgcttccgagggcgattttactgctgtcagagaaattgcaaagtccgttggcaaccgtgacaacccaccaatcatttgtggcttggccagagctctcgaaaaggatatctcaagatgttacgaagccgtcaagcacgcagcatttccaagaatccacaccttcatcgcaacatcagatttgcacatggcgtataaattaaagaagaccagagaggaagtcgttgaaattacaaaggaaacagttacatatgccagaagtttgtgtgaggacgttgagttttctgccgaagatgccatcagatccgatccagacttcctctgtgaggtcttctccgcggctattgaggctggcgcaactactatcaacgttcctgacaccgttggttacaccaccccatcggaatttgcgtcccttattagatatttgaggagaaacgtcagaggtattgatgacgttactatctctgtccacggacacgatgatttgggtatggcagttgccaacttcttgtcagccgttgaaaatggcgcccgtcaaatggagtgtacaatcaacggaatcggtgaaagagcaggcaacgcttctctggaagaagtcgtgatggcattacacgttagaagacaattctacaacgctcgtatgggaaaggacaataaggtggacgcgccgctaactaatatagttcacaaggagatacatcacacctctagaatggtcagtaatttaactggaatgctcgttcagccgaataaggcaattgttggagcaaatgcttttgcacacgaatcgggtatccatcaagacggtgttttaaagcatagacaaacgtacgagatcatggatgcacaatccattggtttgtctgagaactccattgtcttgggtaagcactcaggtagacacgcattcagaacaagattggttaacatgggatacgaggtcacggatgaagagttggaaagagcatttagaagattcaaggagttagctgacattaaaaaggaagtgtcggaggctgatttacaatccttggttaacgatgaggtgcgtttggttaaagaagcagttaagctaactagaattcaaattcaatgtggataccacattattccaactgccacaatcggtttgattttggttgatgatgacgcggaaaagactgttacatcaacgggtactggtcctgttgattctgcctacaatgctatcaaccaagtcattgaagatatcatccatgttactctgctcgaatacaaggtttcatctgtctcaaagggtattgatgcgttaggagaagttgcagtccgcgttcaagacggtgctactggtaaccagtatatcggtgcggcagccaatacggatattgttgtggcctccgttcaagcatatgttaatgcgattaacagatgtcaactcaataataagaagcctaagatccatccacagtatggcaacgcgaattctgtttga

SEQ ID NO: 11 - DNA, 1470 bp, Archaeoglobus fulgidus
  source 1..1470: mol_type = genomic DNA
atgcaagttaagattttggatactactttgagagatggtgaacaaactccaggtgtttctttgtctgttgaacaaaaggttatgattgcagaagcattggataacttgggtgttgatattattgaagcaggtactgcaattgcatctgaaggtgatttccaagcaattaaggaaatttctcaaagaggtttgaacgcagaaatttgttctttcgcaagaattaagagagaagatattgatgcagcagcagatgcaggtgcagaatctattttcatggttgcaccatcttctgatattcatattaacgcaaagttcccaggtaaggatagagattacgttattgaaaagtctgttgaagcaattgaatacgcaaaggaaagaggtttgattgttgaattcggtgcagaagatgcatctagagcagatttggatttcgttattcaattgttcaagagagcagaagaagcaaaggcagatagaattactttcgcagatactgttggtgttttgtctccagaaaagatggaagaaattgttagaaagattaaggcaaaggttaagttgccattggcaattcattgtcatgatgatttcggtttggcaactgcaaacactattttcggtattaaggcaggtgcagaagaattccatggtactattaacggtttgggtgaaagagcaggtaacgcagcaattgaagaagttgttattgcattggaatacttgtacggtattaagactaagattaagaaggaaagattgtacaacacttctaagttggttgaaaagttgtctagagttgttgttccaccaaacaagccaattgttggtgataacgcattcactcatgaatctggtattcatacttctgcattgttcagagatgcaaagtcttacgaaccaatttctccagaagttgttggtagaaagagagttattgttttgggtaagcatgcaggtagagcatctgttgaagcaattatgaacgaattgggttacaaggcaactccagaacaaatgaaggaaattttggcaagaattaaggaaattggtgataagggtaagagagttactgatgcagatgttagaactattattgaaactgttttgcaaattaagagagaaaagaaggttaagttggaagatttggcaattttctctggtaagaacgttatgccaatggcatctgttaagttgaagattgatggtcaagaaagaattgaagcagcagttggtttgggtccagttgatgcagcaattaacgcaattagaagagcaattaaggaattcgcagatattaagttggtttcttaccatgttgatgcaattactggtggtactgatgcattggttgatgttgttgttcaattgaagaaggataacaagattgttactgcaagaggtgcaagaactgatattattatggcatctgttgaagcattcattgaaggtattaacatgttgttctaa

SEQ ID NO: 12 - DNA, 1476 bp, Methanocaldococcus jannaschii
  source 1..1476: mol_type = genomic DNA
atgatggttagaattttcgatactactttgagagatggtgaacaaactccaggtgtttctttgactccaaacgataagttggaaattgcaaagaagttggatgaattgggtgttgatgttattgaagcaggttctgcaattacttctaagggtgaaagagaaggtattaagttgattactaaggaaggtttgaacgcagaaatttgttctttcgttagagcattgccagttgatattgatgcagcattggaatgtgatgttgattctgttcatttggttgttccaacttctccaattcatatgaagtacaagttgagaaagactgaagatgaagttttggaaactgcattgaaggcagttgaatacgcaaaggaacatggtttgattgttgaattgtctgcagaagatgcaactagatctgatgttaacttcttgattaagttgttcaacgaaggtgaaaaggttggtgcagatagagtttgtgtttgtgatactgttggtgttttgactccacaaaagtctcaagaattgttcaagaagattactgaaaacgttaacttgccagtttctgttcattgtcataacgatttcggtatggcaactgcaaacacttgttctgcagttttgggtggtgcagttcaatgtcatgttactgttaacggtattggtgaaagagcaggtaacgcatctttggaagaagttgttgcagcattgaagattttgtacggttacgatactaagattaagatggaaaagttgtacgaagtttctagaattgtttctagattgatgaagttgccagttccaccaaacaaggcaattgttggtgataacgcattcgcacatgaagcaggtattcatgttgatggtttgattaagaacactgaaacttacgaaccaattaagccagaaatggttggtaacagaagaagaattattttgggtaagcattctggtagaaaggcattgaagtacaagttggatttgatgggtattaacgtttctgatgaacaattgaacaagatttacgaaagagttaaggaattcggtgatttgggtaagtacatttctgatgcagatttgttggcaattgttagagaagttactggtaagttggttgaagaaaagattaagttggatgaattgactgttgtttctggtaacaagattactccaattgcatctgttaagttgcattacaagggtgaagatattactttgattgaaactgcatacggtgttggtccagttgatgcagcaattaacgcagttagaaaggcaatttctggtgttgcagatattaagttggttgaatacagagttgaagcaattggtggtggtactgatgcattgattgaagttgttgttaagttgagaaagggtactgaaattgttgaagttagaaagtctgatgcagatattattagagcatctgttgatgcagttatggaaggtattaacatgttgttgaactaa

SEQ ID NO: 13 - DNA, 1587 bp, Geobacter sulfurreducens
  source 1..1587: mol_type = genomic DNA
atgtctttggttaagttgtacgatactactttgagagatggtactcaagcagaagatatttctttcttggttgaagataagattagaattgcacataagttggatgaaattggtattcattacattgaaggtggttggccaggttctaacccaaaggatgttgcattcttcaaggatattaagaaggaaaagttgtctcaagcaaagattgcagcattcggttctactagaagagcaaaggttactccagataaggatcataacttgaagactttgattcaagcagaaccagatgtttgtactattttcggtaagacttgggatttccatgttcatgaagcattgagaatttctttggaagaaaacttggaattgattttcgattctttggaatacttgaaggcaaacgttccagaagttttctacgatgcagaacatttcttcgatggttacaaggcaaacccagattacgcaattaagactttgaaggcagcacaagatgcaaaggcagattgtattgttttgtgtgatactaacggtggtactatgccattcgaattggttgaaattattagagaagttagaaagcatattactgcaccattgggtattcatactcataacgattctgaatgtgcagttgcaaactctttgcatgcagtttctgaaggtattgttcaagttcaaggtactattaacggtttcggtgaaagatgtggtaacgcaaacttgtgttctattattccagcattgaagttgaagatgaagagagaatgtattggtgatgatcaattgagaaagttgagagatttgtctagattcgtttacgaattggcaaacttgtctccaaacaagcatcaagcatacgttggtaactctgcattcgcacataagggtggtgttcatgtttctgcaattcaaagacatccagaaacttacgaacatttgagaccagaattggttggtaacatgactagagttttggtttctgatttgtctggtagatctaacattttggcaaaggcagaagaattcaacattaagatggattctaaggatccagttactttggaaattttggaaaacattaaggaaatggaaaacagaggttaccaattcgaaggtgcagaagcatctttcgaattgttgatgaagagagcattgggtactcatagaaagttcttctctgttattggtttcagagttattgatgaaaagagacatgaagatcaaaagccattgtctgaagcaactattatggttaaggttggtggtaagattgaacatactgcagcagaaggtaacggtccagttaacgcattggataacgcattgagaaaggcattggaaaagttctacccaagattgaaggaagttaagttgttggattacaaggttagagttttgccagcaggtcaaggtactgcatcttctattagagttttgattgaatctggtgataaggaatctagatggggtactgttggtgtttctgaaaacattgttgatgcatcttaccaagcattgttggattctgttgaatacaagttgcataagtctgaagaaattgaaggttctaagaagtaa

SEQ ID NO: 14 - DNA, 1605 bp, Streptomyces coelicolor
  source 1..1605: mol_type = genomic DNA
atgactgcaacttctgaattggatgattctttccatgttttcgatactactttgagagatggtgcacaaagagaaggtattaacttgactgttgcagataagttggcaattgcaagacatttggatgatttcggtgttggtttcattgaaggtggttggccaggtgcaaacccaagagatactgaattcttcgcaagagcaagacaagaaattgatttcaagcatgcacaattggttgcattcggttctactagaagagcaggtgcaaacgcagcagaagatcatcaagttaaggcattgttggattctggtgcacaagttattactttggttgcaaagtctcatgatagacatgttgaattggcattgagaactactttggatgaaaacttggcaatggttgcagatactgtttctcatttgaaggcacaaggtagaagagttttcgttgattgtgaacatttcttcgatggttacagagcaaacccagaatacgcaaagtctgttgttagaactgcatctgaagcaggtgcagatgttgttgttttgtgtgatactaacggtggtatgttgccagcacaaattcaagcagttgttgcaactgttttggcagatactggtgcaagattgggtattcatgcacaagatgatactggttgtgcagttgcaaacactttggcagcagttgatgcaggtgcaactcatgttcaatgtactgcaaacggttacggtgaaagagttggtaacgcaaacttgttcccagttgttgcagcattggaattgaagtacggtaagcaagttttgccagaaggtagattgagagaaatgactagaatttctcatgcaattgcagaagttgttaacttgactccatctactcatcaaccatacgttggtgtttctgcattcgcacataaggcaggtttgcatgcatctgcaattaaggttgatccagatttgtaccaacatattgatccagaattggttggtaacactatgagaatgttggtttctgatatggcaggtagagcatctattgaattgaagggtaaggaattgggtattgatttgggtggtgatagagaattggttggtagagttgttgaaagagttaaggaaagagaattggcaggttacacttacgaagcagcagatgcatctttcgaattgttgttgagagcagaagcagaaggtagaccattgaagtacttcgaagttgaatcttggagagcaattactgaagatagaccagatggttctcatgcaaacgaagcaactgttaagttgtgggcaaagggtgaaagaattgttgcaactgcagaaggtaacggtccagttaacgcattggatagatctttgagagttgcattggaaaagatttacccagaattggcaaagttggatttggttgattacaaggttagaattttggaaggtgttcatggtactcaatctactactagagttttgatttctacttctgatggtactggtgaatgggcaactgttggtgttgcagaaaacgttattgcagcatcttggcaagcattggaagatgcatacacttacggtttgttgagagcaggtgttgcaccagcagaataa

SEQ ID NO: 15 - DNA, 1614 bp, Arabidopsis thaliana
  source 1..1614: mol_type = genomic DNA
atgtctacttctatttctattttcgatactactttgagagatggtactcaaggtgaaggtatttctttgactgcagaagataagattaagattgcattgaagttggatgcattgggtgttcattacattgaaggtggtaacccaggttctaactctaaggatattgaattcttcagaagagcaagagaattgaacttgagagcaaagttgactgcattcggttctactagaagaaagaactctttgtgtgaacaagatgttaacttgttgaacttggtttcttctggtgcaaaggcagcaactttggttggtaagacttgggatttccatgttcatactgcattgcaaactactttggaagaaaacttggcaatgatttacgattctttggcatacttgaagcaacatggtttggaagcaattttcgattctgaacatttcttcgatggtttcaaggcaaacccagattacgcaattgcagcattgagaaaggcacaagaagcaggtgcagattggattgttttgtgtgatactaacggtggtactttgccaaacgaaattcaagatattgttaagcaagttagaaactctattcaagcaccaattggtattcatactcataacgattgtgaattggcagttgcaaacactttggcagcagttactgcaggtgcaagacaaattcaaggtactattaacggttacggtgaaagatgtggtaacgcaaacttgtgttctattttgccaactttgcaattgaagatgggttaccaagttgttactccagaacaattgggttctttgacttctgttgcaagatacgttggtgaaattgcaaacgttgttttgccagttaaccaaccatacgttggtactgcagcattcgcacataagggtggtattcatgtttctgcaattttgaaggattctaagacttacgaacatatttctccagatttggttggtaacaagcaaagagttttggtttctgaattggcaggtcaatctaacattttgtctaaggcacaagaaatgggtttggcagtttctaacgataacgcaaactctagagaagttattgaaaagattaagaacttggaacatcaaggttaccaattcgaaggtgcagatgcatctttggaattgttgttgagagatgcatacggtgatgcagttgaaattttcactgttgaatctttcaagattttgatggaaaagtctccatctggtaacttgactgaagcaattgttaagttgaacgtttctggtcaacaagtttacactgttgcagaaggtaacggtccagttaacgcattggataacgcattgagaaaggcattgactccattctacccagatattaacggtattcatttgtctgattacaaggttagagttttggatgaaaaggatactactgcagcaaaggttagagttttgattgaatctactaacttcaaggaatcttggtctactgttggtgtttcttctaacgttattgaagcatcttgggaagcattgattgattctattagatacgcattgttgggtatgactcaaacttctttctctccagaatctccaagagaatctttgggtttggttaaccattaa

(19) United States
(12) Patent Application Publication
(10) Pub. No.: US 2024/0271168 A1
(43) Pub. Date: Aug. 15, 2024

(54) GENETICALLY MODIFIED YEAST HOSTS AND METHODS FOR PRODUCING CITRAMALATE

(71) Applicants: THE REGENTS OF THE UNIVERSITY OF CALIFORNIA, Oakland, CA (US); IOWA STATE UNIVERSITY RESEARCH FOUNDATION, INC., Ames, IA (US); THE TRUSTEES OF PRINCETON UNIVERSITY, Princeton, NJ (US)

(72) Inventors: Yasuo YOSHIKUNI, Orinda, CA (US); Zong-Yen WU, Berkeley, CA (US); Ping-Hung HSIEH, Berkeley, CA (US); Sudharsan DWARAKNATH, Berkeley, CA (US); Zengyi SHAO, Ames, IA (US); Wan SUN, Ames, IA (US); Joshua D. RABINOWITZ, Princeton, NJ (US); Yihui SHEN, Princeton, CA (US)

(21) Appl. No.: 18/443,118

(22) Filed: Feb. 15, 2024

Related U.S. Application Data
(60) Provisional application No. 63/485,197, filed on Feb. 15, 2023.

(51) Int. Cl. C12P 7/44; C12N 9/10

(52) U.S. Cl. CPC C12P 7/44; C12N 9/1029; C12Y 203/01182

(57) ABSTRACT

The present invention provides for a genetically modified yeast host cell comprising a heterologous citramalate synthase, or multiple copies of a citramalate synthase, and knocked out or reduced in expression, or under conditional expression, for an endogenous or native pyruvate decarboxylase (PDC) gene; a method for constructing the genetically modified yeast host cell, and a method for producing citramalate using the genetically modified yeast host cell.

### CROSS REFERENCE TO RELATED APPLICATIONS

This application claims priority to U.S. Provisional Patent Application Ser. No. 63/485,197, filed Feb. 15, 2023, which is hereby incorporated by reference.

### STATEMENT OF GOVERNMENTAL SUPPORT

The invention was made with government support under Contract Nos. DE-AC02-05CH11231 and DE-SC0018420 awarded by the U.S. Department of Energy. The government has certain rights in the invention.

### REFERENCE TO SEQUENCE LISTING

The instant application contains a Sequence Listing which has been submitted electronically in XML format and is hereby incorporated by reference in its entirety. Said XML copy, created on Feb. 15, 2024, is named “2023-056-03 Sequence Listing.xml” and is 37 kilobytes in size.

### FIELD OF THE INVENTION

This invention relates generally to producing citramalate in yeast.

### BACKGROUND OF THE INVENTION

Methyl methacrylate (MMA) is a building block for poly MMA (PMMA), which is a transparent material known as acrylic glass or plexiglass with the trade names Acrylite® and Plexiglas® (Mahboub et al., 2018). PMMA is an economical alternative to polycarbonate (PC) and has diverse industrial applications (including paints, coatings, electronics, and modifier for polyvinyl chloride [PVC]) (Dixit et al., 2009; Lebeau et al., 2020; Mahboub et al., 2018). PMMA is also commonly used in making prosthetic dental applications, including dentures, denture bases, and artificial teeth (implants) (Frazer et al., 2005; Zafar, 2020). Because of MMA's versatility, its global market demand is expected to grow to USD 8.16 billion by 2025, with a compound annual growth rate of 8.4% (Grand View Research, 2019).

MMA is currently produced from petroleum using chemical processes. The dominant commercial process for MMA is the acetone cyanohydrin (ACH) route. The use of toxic hydrogen cyanide and concentrated acid is a primary concern for the ACH route, as are the negative impacts of co-product waste (ammonium bisulfate) generation and disposal. (Lebeau et al., 2020; Mahboub et al., 2018; Nagai and Ui, 2004). Although the industry has improved the process significantly, even the safer and more-sustainable alternatives recently developed are still energy-intensive, and therefore contribute excessively to greenhouse gas emission. For example, the LiMA process, milder than others, emits 2.6 t-CO₂/t-MMA (Mahboub et al., 2018).

Producing MMA from renewable resources may be a more attractive alternative. Semisynthesis (a combination of biological and chemical processes) may be the best strategy for MMA production, as MMA is toxic to cells because of its lipophilicity and reactivity with cellular components, and no enzyme is currently known to directly catalyze the formation of MMA (Curson et al., 2014; Webb et al., 2018). Diverse metabolites have been proposed as precursors for MMA production (Lebeau et al., 2020). Among them, the most promising approach may be to use di- and tricarboxylic acid metabolites as precursors. In particular, citramalate, a dicarboxylic acid, is selected as a target for semisynthesis because it can easily be converted to methacrylic acid (MA), a precursor for MMA, via base-catalyzed decarboxylation and dehydration in hot pressurized water (Johnson et al., 2015; Wu and Eiteman, 2016). MA is then converted into MMA through esterification in the presence of methanol and an acid catalyst (Lebeau et al., 2020).

Citramalate is a common metabolite found in diverse organisms as an intermediate of the isoleucine biosynthesis pathway (Risso et al., 2008; Sugimoto et al., 2021). The key enzyme for citramalate synthesis is citramalate synthase (CimA, EC 2.3.1.182), which catalyzes condensation of the central metabolites pyruvate and acetyl-CoA to generate citramalate (Howell et al., 1999). An E. coli strain has been engineered to produce citramalate. This strain carried an exogenous citramalate synthase gene (cimA) with the genes for lactate dehydrogenase (ldh) and pyruvate formate lyase (pfl) deleted. The low toxicity of citramalate compared to many other organic acids helped increase its production significantly. Fed-batch fermentation using this E. coli strain achieved a titer of 82 g/L, a productivity of 1.85 g L⁻¹ hr⁻¹, and a conversion yield of 0.48 wt % (Webb et al., 2018).

One major bottleneck for this process, however, is that a neutralization step is required. At a large scale of production, a cheap alkali source, lime (CaCO₃), is generally used for the neutralization, which results in high CO₂ emission. Additionally, the media must be reacidified with H₂SO₄ to convert the salt form to the undissociated form of citramalate, resulting in formation of a large amount of gypsum (CaSO₄) as a byproduct that needs to be properly disposed. The technoeconomic assessment and life cycle assessment for organic acid production suggest that neutralization and acidification steps increase both process cost and environmental footprint by 30% (Bhagwat et al., 2021). Low-pH fermentation using acid-tolerant microbes are therefore a better process for citramalate production.

Acknowledging the benefits of low-pH fermentation, the US Department of Energy's Center for Bioenergy and Bioproduct Innovations (CABBI) selected Issatchenkia orientalis as its flagship strain for organic acid production because of its ability to tolerate extremely low pH. I. orientalis has already been engineered to produce some organic acids, including D-xylonic acid (Toivari et al., 2013), succinic acid (Xiao et al., 2014), D-lactic acid (Park et al., 2018), itaconic acid (Sun et al., 2020), and 3-hydroxypropionic acid (Bindel, 2016). With recent advances in genetic and genomic engineering tools (e.g., plasmid, promoters, terminators, and CRISPR-Cas9 system) (Cao et al., 2020; Tran et al., 2019) and a genome-scale metabolic model, ilsor850 (Suthers et al., 2020), I. orientalis is becoming a more amenable strain for metabolic engineering.

### SUMMARY OF INVENTION

The present invention provides for a genetically modified yeast host cell comprising a heterologous citramalate synthase, or multiple copies of a citramalate synthase, and knocked out or reduced in expression, or under conditional expression, for an endogenous or native pyruvate decarboxylase (PDC) gene.

In some embodiments, the genetically modified yeast host cell is an Ascomycota cell. In some embodiments, the Ascomycota cell is a Saccharomyces cell. In some embodiments, the Saccharomyceses cell is a Saccharomycesles cell. In some embodiments, the Saccharomycesles cell is a Pischiaceae cell. In some embodiments, the Pischiaceae cell is an Issatchenkia cell. In some embodiments, the Issatchenkia cell is an Issatchenkia hanoiensis or Issatchenkia orientalis cell.

In some embodiments, the heterologous citramalate synthase is a citramalate synthase from any strain described herein, such as in Table 4. In some embodiments, the heterologous citramalate synthase has an enzymatic activity higher or greater an endogenous or native citramalate synthase of the genetically modified yeast host cell.

In some embodiments, the citramalate synthase is a homologous enzyme thereof, comprising an amino acid sequence having at least about 70%, 75%, 80%, 85%, 90%, 95%, or 99% amino acid sequence identity with any citramalate synthase from any strain described herein, such as in Table 4. In some embodiments, the homologous enzyme of citramalate synthase has an amino acid sequence not found in nature. In some embodiments, the homologous enzyme of citramalate synthase has an amino acid sequence comprising one or more conserved amino acids, each in its corresponding position, that is found conserved among the citramalate synthases described herein, such as in Table 4.

In some embodiments, the genetically modified yeast host cell comprises: (a) a nucleic acid encoding the heterologous citramalate synthase operatively linked to a promoter, or (b) one or more nucleic acids encoding one or a plurality of a citramalate synthase gene(s), heterologous or native to the genetically modified yeast host cell, or both, wherein each citramalate synthase gene is operatively linked to a promoter; wherein the or each promoter is capable of expressing the citramalate synthase in the genetically modified yeast host cell. In some embodiments, the nucleic acid encoding the heterologous citramalate synthase is codon optimized specifically for the genetically modified yeast host cell.

The present invention provides for a composition comprising a culture medium comprising a genetically modified yeast host cell of the present invention. In some embodiments, the culture medium has a low pH. In some embodiments, the pH is about 2.0, 2.5, 3.0, 3.5, 4.0, 4.5, 5.0, 5.5, 6.0, or any range of two preceding pH values. In some embodiments, the pH is about 2.0 to 6.0, or about 2.0 to 5.5. In some embodiments, the pH is about 3.0 to 6.0, or about 3.0 to 5.5. In some embodiments, the genetically modified yeast host cell is producing citramalate. The genetically modified yeast host cell is capable of tolerating, growing or being cultured in the low pH culture medium of the present invention.

The present invention provides for a method for constructing the genetically modified yeast host cell of the present invention, comprising: (a) introducing a nucleic acid encoding citramalate synthase operatively linked to a promoter in a yeast host cell, and (b) optionally deleting, knocking out, or reducing the expression for an endogenous or native pyruvate decarboxylase (PDC) gene.

The present invention provides for a method for producing citramalate, comprising: (a) introducing the genetically modified yeast host cell of the present invention to a culture medium, (b) growing or culturing the genetically modified yeast host such that the genetically modified yeast host produce citramalate, and (c) optionally separating the citramalate from the genetically modified yeast host cell and/or the culture medium.

In some embodiments, the method further comprises: converting the citramalate into methacrylic acid (MA), optionally converting the MA into methyl methacrylate (MMA), and optionally polymerizing MMA into poly MMA (PMMA). Citramalate can be converted into MA via base-catalyzed decarboxylation and dehydration in hot pressurized water. MA can be converted in MMA through esterification in the presence of methanol and an acid catalyst.

In some embodiments, the method produces citramalate with a yield or rate about equal to or higher than any yield or rate described herein, or within a range of yield or rate of about any two yields or rate described herein.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Tolerance of I. orientalis SD108 to various pH values and citramalate concentrations. (A) Growth curve measured in YNB broth at various pH values (1.5, 2.0, 2.5, 3.0, 3.5, and 5.5) at 30° C. (B) Growth curve measured in YNB broth with various citramalate concentrations at pH 3.0. The data is collected in biological triplicate. The shaded areas indicate the standard deviation of the triplicate measurements.

FIG. 2. Identification of a more active cimA variant for citramalate production in I. orientalis SD108. (A) Schematic representation of the pathway for citramalate production. Citramalate is formed from condensation between pyruvate and acetyl-CoA. (B) The SSN analysis was used for target gene selection. The cimA variants that have been previously characterized are denoted with blue circles. The cimA variants of eukaryotic origin are denoted with green circles. The orange circles indicate the cimA variants randomly selected from different clades. The number in each dot represents the synthetic cimA gene ID. The sequences of those genes are listed in Table 4 and 5. (C) Citramalate production from I. orientalis SD108 expressing five active genes in a plasmid with two different codon optimization strategies using BOOST (Oberortner et al., 2017). The black bars indicate genes optimized with “balanced” codon usage. The gray bars represent genes optimized with “mostly used” codon usage. These samples were measured at 48 h after cultivation. All experiments were done in technical triplicate.

FIG. 3. Random integration of cimA into I. orientalis and citramalate production. (A) The schematics showing the PiggyBac transposon-mediated genome integration of cimA. The plasmid containing the cimA transposon integration cassette (ITR-GFP-CimA-LEU-ITR) and PiggyBac transposase gene (hpB7) was transformed into I. orientalis SD108. Catalyzed by the PiggyBac transposase, this integration cassette was randomly integrated into the ‘TTAA’ sites in the I. orientalis genome. This system can also integrate multiple copies of the integration cassette. (B) Comparison of citramalate production from the cimA integration variants. The four best producers, SB814 (Strain ID #12), SB815 (Strain ID #20), SB816 (Strain ID #33), and SB817 (Strain ID #40) are highlighted in yellow. The experiments were carried out with single replicate. Citramalate production levels at 120 h were compared.

FIG. 4. Citramalate production from the cimA-integrated I. orientalis strains in SC medium. Strains were cultivated in SC containing 50 g/L glucose at 30° C. at 250 rpm for 72 hours. (A) citramalate production, (B) glucose consumption, and (C) growth. Byproducts shown are (D) pyruvate, (E) glycerol, and (F) EtOH, measured using LC-MS. All experiments were performed in biological triplicate.

FIG. 5. Citramalate production from the cimA-integrated I. orientalis strains in YPD medium. Strains were cultivated in YPD containing 50 g/L glucose at 30° C. at 250 rpm for 72 hours. (A) citramalate production, (B) glucose consumption, and (C) growth. Byproducts shown are (D) pyruvate, (E) glycerol, and (F) EtOH, measured using LC-MS. All experiments were performed in biological triplicate.

FIG. 6. Plasmid map of cimA expression vector pZF_EcoR1_TDH3p. This plasmid has a ColE1 origin of replication and an ampicillin resistance gene for cloning work in E. coli. An S. cerevisiae autonomously replicating sequence (ARS) is used for maintaining the plasmid in I. orientalis. This plasmid also contains the I. orientalis uracil auxotrophic selection marker (URA3), and a unique EcoR1 site between TDH3 promoter and ENO2 terminator, allowing insertion of the synthetic gene.

FIG. 7. The plasmid map of pWS-URA-hPB7-GFP-CimA-LEU.

FIG. 8. Genomic version green fluorescence protein (GFP) expression in I. orientalis.

FIG. 9. pH values of YPD medium for cultivating SB814. The pH dropped rapidly to 3.2 within 24 hours and maintained between 3.2 and 3.4 until the endpoint at 96 hours of cultivation.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to “molecules” includes a plurality of a molecule species as well as a plurality of molecules of different species.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

### REFERENCES CITED

Altschul, S. F., Gish, W., Miller, W., Myers, E. W., Lipman, D. J., 1990. Basic local alignment search tool. J. Mol. Biol. 215, 403-410.
Bhagwat, S. S., Li, Y., Cortés-Peña, Y. R., Brace, E. C., Martin, T. A., Zhao, H., Guest, J. S., 2021. Sustainable Production of Acrylic Acid via 3-Hydroxypropionic Acid from Lignocellulosic Biomass. ACS Sustainable Chem. Eng. 9, 16659-16669.
Bindel, M., 2016. 3-hydroxypropionic acid production by recombinant yeasts. U.S. Pat. No. 9,365,875.
Cao, M., Fatma, Z., Song, X., Hsieh, P.-H., Tran, V. G., Lyon, W. L., Sayadi, M., Shao, Z., Yoshikuni, Y., Zhao, H., 2020. A genetic toolbox for metabolic engineering of Issatchenkia orientalis. Metab. Eng. 59, 87-97.
Chambers, M. C., Maclean, B., Burke, R., Amodei, D., Ruderman, D. L., Neumann, S., Gatto, L., Fischer, B., Pratt, B., Egertson, J., Hoff, K., Kessner, D., Tasman, N., Shulman, N., Frewen, B., Baker, T. A., Brusniak, M.-Y., Paulse, C., Creasy, D., Flashner, L., Kani, K., Moulding, C., Seymour, S. L., Nuwaysir, L. M., Lefebvre, B., Kuhlmann, F., Roark, J., Rainer, P., Detlev, S., Hemenway, T., Huhmer, A., Langridge, J., Connolly, B., Chadick, T., Holly, K., Eckels, J., Deutsch, E. W., Moritz, R. L., Katz, J. E., Agus, D. B., MacCoss, M., Tabb, D. L., Mallick, P., 2012. A cross-platform toolkit for mass spectrometry and proteomics. Nat. Biotechnol. 30, 918-920.
Chen, I.-M. A., Chu, K., Palaniappan, K., Pillay, M., Ratner, A., Huang, J., Huntemann, M., Varghese, N., White, J. R., Seshadri, R., Smirnova, T., Kirton, E., Jungbluth, S. P., Woyke, T., Eloe-Fadrosh, E. A., Ivanova, N. N., Kyrpides, N. C., 2019. IMG/M v.5.0: an integrated data management and comparative analysis system for microbial genomes and microbiomes. Nucleic Acids Res. 47, D666-D677.
Curson, A. R. J., Burns, O. J., Voget, S., Daniel, R., Todd, J. D., McInnis, K., Wexler, M., Johnston, A. W. B., 2014. Screening of metagenomic and genomic libraries reveals three classes of bacterial enzymes that overcome the toxicity of acrylate. PLOS One 9, e97660.
Da Silva, N. A., Srikrishnan, S., 2012. Introduction and expression of genes for metabolic engineering applications in Saccharomyces cerevisiae. FEMS Yeast Res. 12, 197-214.
Dixit, M., Mathur, V., Gupta, S., Baboo, M., Sharma, K., Saxena, N. S., Others, 2009. Investigation of miscibility and mechanical properties of PMMA/PVC blends. J. Optoelectron. Adv. Mater. Rapid Commun 3, 1099-1105.
Flagfeldt, D. B., Siewers, V., Huang, L., Nielsen, J., 2009. Characterization of chromosomal integration sites for heterologous gene expression in Saccharomyces cerevisiae. Yeast 26, 545-551.
Frazer, R. Q., Byron, R. T., Osborne, P. B., West, K. P., 2005. PMMA: an essential material in medicine and dentistry. J. Long Term Eff. Med. Implants 15, 629-639.
Gerlt, J. A., Bouvier, J. T., Davidson, D. B., Imker, H. J., Sadkhin, B., Slater, D. R., Whalen, K. L., 2015. Enzyme Function Initiative-Enzyme Similarity Tool (EFI-EST): A web tool for generating protein sequence similarity networks. Biochim. Biophys. Acta 1854, 1019-1037.
Grand View Research, 2019. PMMA Market Size Worth $8.16 Billion By 2025| CAGR: 8.4% [WWW Document]. Webpage at: grandviewresearch.com/press-release/global-polymethyl-methacrylate-pmma-industry (accessed 12.19.21).
Grigoriev, I. V., Nikitin, R., Haridas, S., Kuo, A., Ohm, R., Otillar, R., Riley, R., Salamov, A., Zhao, X., Korzeniewski, F., Smirnova, T., Nordberg, H., Dubchak, I., Shabalov, I., 2014. MycoCosm portal: gearing up for 1000 fungal genomes. Nucleic Acids Res. 42, D699-704.
Howell, D. M., Xu, H., White, R. H., 1999. (R)-citramalate synthase in methanogenic archaea. J. Bacteriol. 181, 331-333.
Johnson, D. W., Eastham, G. R., Poliakoff, M., Huddle, T. A., 2015. Method of producing arcylic and methacrylic acid. U.S. Pat. No. 8,933,179.
Lebeau, J., Efromson, J. P., Lynch, M. D., 2020. A Review of the Biotechnological Production of Methacrylic Acid. Frontiers in Bioengineering and Biotechnology 8, 207.
Li, X., Burnight, E. R., Cooney, A. L., Malani, N., Brady, T., Sander, J. D., Staber, J., Wheelan, S. J., Joung, J. K., McCray, P. B., Jr, Bushman, F. D., Sinn, P. L., Craig, N. L., 2013. piggyBac transposase tools for genome engineering. Proc. Natl. Acad. Sci. U.S.A. 110, E2279-87.
Mahboub, M. J. D., Dubois, J.-L., Cavani, F., Rostamizadeh, M., Patience, G. S., 2018. Catalysis for the synthesis of methacrylic acid and methyl methacrylate. Chem. Soc. Rev. 47, 7703-7738.
Meadows, A. L., Hawkins, K. M., Tsegaye, Y., Antipov, E., 2016. Rewriting yeast central carbon metabolism for industrial isoprenoid production. Nature.
Nagai, K., Ui, T., 2004. Trends and future of monomer-MMA technologies. Sumitomo Chem 2, 4-13.
Nakamura, yasukazu, 2007. Codon Usage Database [WWW Document]. Codon Usage Database. Webpage at: kazusa.or.jp/codon/
Nielsen, J., 2014. Synthetic biology for engineering acetyl coenzyme A metabolism in yeast. MBio 5, e02153.
Oberortner, E., Cheng, J.-F., Hillson, N. J., Deutsch, S., 2017. Streamlining the Design-to-Build Transition with Build-Optimization Software Tools. ACS Synth. Biol. 6, 485-496.
Park, H. J., Bae, J.-H., Ko, H.-J., Lee, S.-H., Sung, B. H., Han, J.-I., Sohn, J.-H., 2018. Low-pH production of d-lactic acid using newly isolated acid tolerant yeast Pichia kudriavzevii NG7. Biotechnol. Bioeng. 115, 2232-2242.
Plotkin, J. B., Kudla, G., 2011. Synonymous but not the same: the causes and consequences of codon bias. Nat. Rev. Genet. 12, 32-42.
Risso, C., Van Dien, S. J., Orloff, A., Lovley, D. R., Coppi, M. V., 2008. Elucidation of an alternate isoleucine biosynthesis pathway in Geobacter sulfurreducens. J. Bacteriol. 190, 2266-2274.
Rodriguez, J. R., Paterson, B. M., 1990. Yeast myosin heavy chain mutant: maintenance of the cell type specific budding pattern and the normal deposition of chitin and cell wall components requires an intact myosin heavy chain gene. Cell Motil. Cytoskeleton 17, 301-308.
Shannon, P., Markiel, A., Ozier, O., Baliga, N. S., Wang, J. T., Ramage, D., Amin, N., Schwikowski, B., Ideker, T., 2003. Cytoscape: a software environment for integrated models of biomolecular interaction networks. Genome Res. 13, 2498-2504.
Shao, Z., Luo, Y., Zhao, H., 2012. DNA assembler method for construction of zeaxanthin-producing strains of Saccharomyces cerevisiae. Methods Mol. Biol. 898, 251-262.
Shao, Z., Zhao, H., 2014. Manipulating natural product biosynthetic pathways via DNA assembler. Curr. Protoc. Chem. Biol. 6, 65-100.
Shao, Z., Zhao, Hua, Zhao, Huimin, 2009. DNA assembler, an in vivo genetic method for rapid construction of biochemical pathways. Nucleic Acids Res. 37, e16.
Sugimoto, N., Engelgau, P., Jones, A. D., Song, J., Beaudry, R., 2021. Citramalate synthase yields a biosynthetic pathway for isoleucine and straight- and branched-chain ester formation in ripening apple fruit. Proc. Natl. Acad. Sci. U.S.A. 118.
Sun, W., Vila-Santa, A., Liu, N., Prozorov, T., Xie, D., Faria, N. T., Ferreira, F. C., Mira, N. P., Shao, Z., 2020. Metabolic engineering of an acid-tolerant yeast strain Pichia kudriavzevii for itaconic acid production. Metab Eng Commun 10, e00124.
Suthers, P. F., Dinh, H. V., Fatma, Z., Shen, Y., Chan, S. H. J., Rabinowitz, J. D., Zhao, H., Maranas, C. D., 2020. Genome-scale metabolic reconstruction of the non-model yeast Issatchenkia orientalis SD108 and its application to organic acids production. Metab Eng Commun 11, e00148.
Toivari, M., Vehkomaki, M.-L., Nygård, Y., Penttilä, M., Ruohonen, L., Wiebe, M. G., 2013. Low pH D-xylonate production with Pichia kudriavzevii. Bioresour. Technol. 133, 555-562.
Tran, V. G., Cao, M., Fatma, Z., Song, X., Zhao, H., 2019. Development of a CRISPR/Cas9-Based Tool for Gene Deletion in Issatchenkia orientalis. mSphere 4.
Vemuri, G. N., Eiteman, M. A., McEwen, J. E., Olsson, L., Nielsen, J., 2007. Increasing NADH oxidation reduces overflow metabolism in Saccharomyces cerevisiae. Proc. Natl. Acad. Sci. U.S.A. 104, 2402-2407.
Wagner, J. M., Williams, E. V., Alper, H. S., 2018. Developing a piggyBac Transposon System and Compatible Selection Markers for Insertional Mutagenesis and Genome Engineering in Yarrowia lipolytica. Biotechnol. J. 13, e1800022.
Webb, J. P., Arnold, S. A., Baxter, S., Hall, S. J., Eastham, G., Stephens, G., 2018. Efficient bio-production of citramalate using an engineered Escherichia coli strain. Microbiology 164, 133-141.
Wu, X., Eiteman, M. A., 2016. Production of citramalate by metabolically engineered Escherichia coli. Biotechnol. Bioeng. 113, 2670-2675.
Xiao, H., Shao, Z., Jiang, Y., Dole, S., Zhao, H., 2014. Exploiting Issatchenkia orientalis SD108 for succinic acid production. Microb. Cell Fact. 13, 121.
Yant, S. R., Kay, M. A., 2003. Nonhomologous-end-joining factors regulate DNA repair fidelity during Sleeping Beauty element transposition in mammalian cells. Mol. Cell. Biol. 23, 8505-8518.
Yu, J., Marshall, K., Yamaguchi, M., Haber, J. E., Weil, C. F., 2004. Microhomology-dependent end joining and repair of transposon-induced DNA hairpins by host factors in Saccharomyces cerevisiae. Mol. Cell. Biol. 24, 1351-1364.
Yusa, K., Zhou, L., Li, M. A., Bradley, A., Craig, N. L., 2011. A hyperactive piggyBac transposase for mammalian applications. Proc. Natl. Acad. Sci. U.S.A. 108, 1531-1536.
Zafar, M. S., 2020. Prosthodontic Applications of Polymethyl Methacrylate (PMMA): An Update. Polymers 12.
Zhao, Y., Yao, Z., Ploessl, D., Ghosh, S., Monti, M., Schindler, D., Gao, M., Cai, Y., Qiao, M., Yang, C., Cao, M., Shao, Z., 2020. Leveraging the Hermes Transposon to Accelerate the Development of Nonconventional Yeast-based Microbial Cell Factories. ACS Synth. Biol. 9, 1736-1752.
Xiao, Han, Zengyi Shao, Yu Jiang, Sudhanshu Dole, and Huimin Zhao. 2014. “Exploiting Issatchenkia Orientalis SD108 for Succinic Acid Production.” Microbial Cell Factories 13 (August): 121.
Howell, D. M., Xu, H., White, R. H., 1999. (R)-citramalate synthase in methanogenic archaea. J. Bacteriol. 181, 331-333.
Zhao, Yuxin, Zhanyi Yao, Deon Ploessl, Saptarshi Ghosh, Marco Monti, Daniel Schindler, Meirong Gao, et al. 2020. “Leveraging the Hermes Transposon to Accelerate the Development of Nonconventional Yeast-Based Microbial Cell Factories.” ACS Synthetic Biology 9 (7): 1736-52.

### Example 1

### Metabolic Engineering of Low-pH-Tolerant Non-Model Yeast, Issatchenkia orientalis, for Production of Citramalate

Methyl methacrylate (MMA) is an important petrochemical with many applications. However, its manufacture has a large environmental footprint. Combined biological and chemical synthesis (semisynthesis) may be a promising alternative to reduce both cost and environmental impact, but strains that can produce the MMA precursor (citramalate) at low pH are required. A non-conventional yeast, Issatchenkia orientalis, may prove ideal, as it can survive extremely low pH. Here, we demonstrate the engineering of I. orientalis for citramalate production. Using sequence similarity network analysis and subsequent DNA synthesis, we selected a more active citramalate synthase gene (cimA) variant for expression in I. orientalis. We then adapted a piggyBac transposon system for I. orientalis that allowed us to simultaneously explore the effects of different cimA gene copy numbers and integration locations. A batch fermentation showed the genome-integrated-cimA strains produced 2.0 g/L citramalate in 48 hours and a yield of up to 7% mol citramalate/mol consumed glucose. These results demonstrate the potential of I. orientalis as a chassis for citramalate production.

In this study, we attempted to engineer I. orientalis for production of citramalate. We first screened cimA genes and identified a more active variant in I. orientalis. To stably integrate this cimA gene variant into I. orientalis's genome, we employed a hyperactive piggyBac transposase system (Li et al., 2013; Wagner et al., 2018; Yusa et al., 2011) and generated a cimA integration library. This system allows us to explore the effect of both various cimA integration locations and different numbers of cimA integration copy on citramalate production. Subsequent screening of this library identified a citramalate producer that was drastically better than its plasmid-based counterpart.

### Materials and Methods

### Strains, Media, and Chemicals

The strains used in this study are listed in Table 2. Dr. Hulmin Zhao (University of Illinois Urbana-Champaign) kindly provided I. orientalis SD108, I. orientalis SD108 ΔURA3, and I. orientalis SD108 ΔURA3 ΔLEU2, which were used as hosts for citramalate production. S. cerevisiae YSG50 (MATα, ADE2-1, ADE3422, URA3-1, HIS3-11,15, TRP1-1, LEU2-3,112, and CAN1-100) was the host for plasmid assembly using the DNA assembler (Shao et al., 2012; Shao and Zhao, 2014). E. coli strain BW25141 was used for plasmid propagation. Yeast extract-peptone-dextrose (YPD) medium containing 1% yeast extract, 2% peptone, and 2% dextrose was used to grow yeast strains. Yeast nitrogen base with amino acids (YNB) containing 2% glucose was used for pH and citramalate tolerance analysis. Synthetic complete dropout medium without uracil (SC-URA) or leucine (SC-LEU) containing 0.5% ammonium sulfate, 0.16% yeast nitrogen base without amino acid or ammonium sulfate, CSM-URA/LEU (added according to manufacturer's instruction), 0.043% adenine hemisulfate, and 2% dextrose were used to select the yeast transformants containing the auxotrophic selection plasmid. 0.1 mg/mL 5-fluoroorotic acid (5-FOA, GoldBio, St Louis, MO) was added to the SC-LEU plate for URA3 counterselection unless otherwise stated. Luria-Bertani (LB) broth supplemented with 100 μg/mL ampicillin was used to grow E. coli strains. The Wizard Genomic DNA Purification Kit was purchased from Promega (Madison, WI). FastDigest restriction enzymes were purchased from Thermo Fisher Scientific (Waltham, MA). Q5 DNA polymerase was purchased from New England Biolabs (Ipswich, MA). The QIAprep Spin Plasmid Mini-prep Kit and RNeasy Mini Kit were purchased from Qiagen (Valencia, CA). Zymoprep Yeast Plasmid Miniprep II Kit was purchased from Zymo Research (Irvine, CA). Oligonucleotides and gBlocks were synthesized by Integrated DNA Technologies (Coralville, IA).

### pH and Citramalate Tolerance Analysis

To test the pH tolerance of I. orientalis SD108, we first streaked the glycerol stock of this strain on a YPD plate and grew it overnight at 30° C. A single colony was picked up from the plate and inoculated in 2 mL YNB broth containing 2% glucose with an initial pH of 5.3, then grown overnight at 30° C. with constant shaking at 250 rpm on a platform shaker. The 2 mL seed culture was pelleted and diluted in the same fresh YNB broth (containing 2% glucose at pH 5.3) with an OD₆₀₀ of 1.5, and then grown at 30° C. with constant shaking at 250 rpm on a platform shaker for 2 h. Then the culture was pelleted and diluted to an OD₆₀₀ of 0.1 in same YNB/glucose broth at various pH values (1.5, 2.0, 2.5, 3.0, 3.5, and 5.5), adjusted by HCl. 200 μL cultures from each condition were added to the wells, and OD₆₀₀ was measured every 30 min for 60.5 h at 30° C. with constant shaking in a plate reader. The same protocol was applied to test the tolerance of citramalate at 40 g/L and 80 g/L at pH 3.0 with various concentrations of citramalate (Sigma-Aldrich SKU-27455 Potassium citramalate monohydrate). 200 μL cultures from each condition were added to the wells, and OD₆₀₀ was measured every 30 min for 96.5 h at 30° C. with constant shaking in a plate reader.

### CimA Sequence Similarity Network Construction and Target Gene Selection

The CimA sequence similarity network (SSN) was constructed using the Enzyme Function Initiative-Enzyme Similarity Tool (EFI-EST) (Gerlt et al., 2015). A well-studied CimA from Methanocaldococcus jannaschii (UniProt ID Q58787) was used as the query for SSN construction. Cytoscape was used to visualize the SSN (Shannon et al., 2003). An overview of the CimA SSN used in this study is provided in Table 3. We first selected genes that had been reported in the literature and subsequently included genes with eukaryotic origins. We also chose the sequences randomly from different clusters in which the UniProt annotation score was greater than 3. Among the 10 selected genes, we optimized the codon usage using JGI Build-Optimization Software Tools (BOOST) (Oberortner et al., 2017) with different strategies to minimize the chance that the codon optimization would accidentally design sequences resulting in poor expression. We then purchased the synthetic gene fragments from Twist Bioscience. We synthesized these genes with “balanced” and “mostly used” strategies (Table 4) in which each of the DNA sequences statistically resembles the I. orientalis codon usage table (Nakamura, 2007). The least-used codons were eliminated (Table 5).

### Plasmid Construction

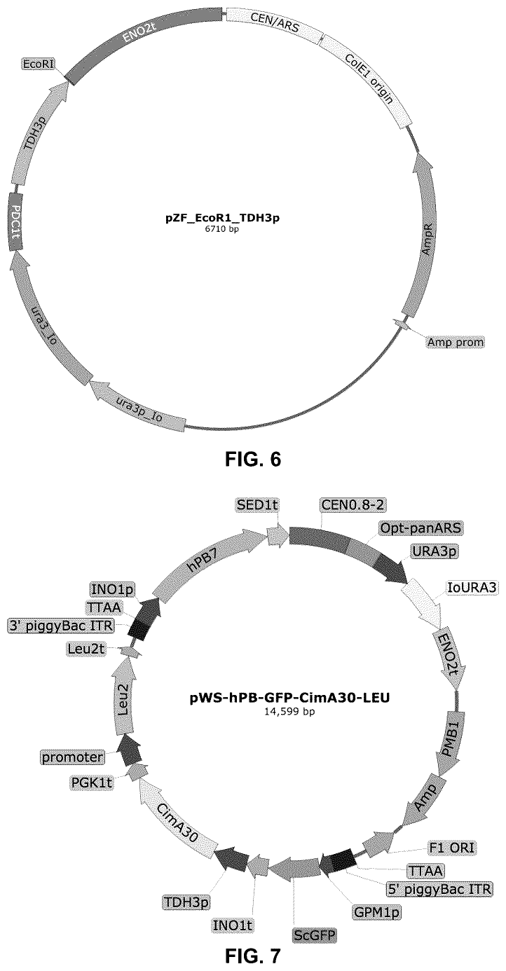

The plasmids used in this study are listed in Table 2. The cimA expression vector pZF_TDH3p with URA3 selection marker was used for identifying an I. orientalis-compatible cimA gene. The TDH3 promoter drives the synthetic cimA gene; ENO2 was used as the terminator in this plasmid. We introduced an EcoR1 cutting site between the TDH3 promoter and the ENO2 terminator to generate pZF_EcoR1_TDH3, which allowed cloning of the synthetic cimA gene into pZF_EcoR1_TDH3p (Supplementary FIG. 6). To construct the plasmid for cimA genome integration, we amplified the centromere-like sequence, autonomously replicating sequence (ARS), and URA3 cassette from pScARS/CEN-L and assembled them as the backbone (Cao et al., 2020). Both pZF_TDH3p and pSsARS/CEN-L vectors were provided by Dr. Hulmin Zhao's group from the University of Illinois at Urbana-Champaign. We codon-optimized and synthesized the hyperactive piggyBac transposase hPB7 variant (I30V, G165S, S103P, M282V, S509G/N570S, and N538K) (Yusa et al., 2011), and then cloned it under the control of the INO1 promoter and the SED1 terminator. We enclosed the cimA cassette flanked by a green fluorescence protein (GFP) cassette and a LEU cassette in the two piggyBac inverted terminal repeats (ITRs) sequence (5′ITR-GFP-CimA-LEU-3′ITR) and assembled them together with an E. coli helper fragment amplified from pRS416. The assembly was performed in S. cerevisiae YSG50 via DNA assembler (Shao et al., 2012; Shao and Zhao, 2014). The plasmid was confirmed by restriction digestion and sequencing and named pWS-URA-hPB7-GFP-CimA-LEU (Supplementary FIG. 7).

### Purification and In Vitro Characterization of CimA Variants

I. orientalis cells expressing citramalate synthase variants (attached with a C-terminal His-tag) were grown in SC-URA medium. A 5 mL overnight culture was used to inoculate 100 mL of media in 500 mL flasks to a starting OD₆₀₀ of 0.1. Cultures were grown at 30° C. at 200 rpm for 20 hours. The suspensions were pelleted, washed, and lysed using a CelLytic Y lysis reagent (Sigma-Aldrich) that included 10 mM DTT, according to the manufacturer's instructions. The lysate was passed through a desalting column and purified by Ni-NTA spin column chromatography (Qiagen). Enzyme concentration was measured by Bradford Assay using the Pierce Coomassie Protein Assay Kit (Thermo Scientific). The specific activity of citramalate biosynthesis in vitro was measured by incubating 0.1 μM enzyme, 1 mM acetyl-CoA, and 20 mM sodium pyruvate in 100 mM TES buffer at pH 7.5 at 30° C. for 50 min following a procedure reported earlier (Howell et al., 1999).

### Strain Construction

The strains used in this study are listed in Table 2. To identify the compatibility of the synthetic cimA gene in I. orientalis, we transformed the cimA expression plasmids into I. orientalis SD108 ΔURA3 using the Frozen-EZ Yeast Transformation II Kit (Zymo Research) and following the manufacturer's instructions. After the transformation, the cells were washed with sterile distilled water once and resuspended in 500 μL SC-URA broth, then cultivated at 30° C. for 2 h. 150 μL of cell culture was spread across the surface of the SC-URA agar plate, and then incubated for 48 h at 30° C. Colonies were randomly picked for further PCR confirmation. To construct genome-integrated-cimA strains using piggyBac-mediated transposition, about 1 μg of pWS-URA-hPB7-GFP-CimA-LEU was transformed into I. orientalis SD108 ΔURA3 ΔLEU2 by electroporation at 2.0 kV and selected on an SC-LEU plate. To enable efficient transposase expression and DNA transposition, the colonies that appeared on the plate were washed into approximately 10 mL SC-LEU broth and grown at 30° C. at 250 rpm for 3 days according to a previous transposition study in Yarrowia lipolytica (Wagner et al., 2018). The cell culture was then diluted and spread on both SC-LEU and SC-LEU+5FOA plates. Colonies that grew on SC-LEU+5FOA plates were collected as the genome-integrated-cimA strain library.

### Flow Cytometry

50 single colonies from the genome-integrated-cimA strain library were picked from the SC-LEU+FOA plate and grown in 2 mL SC-LEU medium for 24 to 36 h. Then 10 μL of the cell culture was diluted in 10 mM phosphate-buffered saline (pH 7.4) and analyzed by flow cytometry at 488 nm with a FACSCanto flow cytometer (BD Biosciences, San Jose, CA) for GFP. BD FACSCanto clinical software was used to evaluate the flow cytometry data.

### Plasmid Removal

To make sure there was no plasmid left in the genome-integrated-cimA strain, four top citramalate-producing strains were grown in 2 mL SC-LEU broth supplemented with 2 g/L FOA for 2 days, then spread on SC-LEU+FOA plates. After colonies were seen on plates, single colonies were picked and duplicated on both SC-URA and SC-LEU+FOA plates. Colonies that could only grow on SC-LEU+FOA plates were our final genome-integrated-cimA strains. To ensure that the strains only have stably expressed genomic cimA, 5-FOA counterselection was performed to cure the piggyBac-expressing plasmid. It is worth pointing out that our final four top producer SB814, SB815, SB816, and SB817 were generated through 2-step counterselection. During the construction of the cimA-integrated I. orientalis strain, the transformants on SC-LEU plates were re-streaked on SC-LEU+5FOA plates. Presumably, the original plasmids or the re-ligated plasmids post-transposition were cured. However, the re-streaked cells were still able to grow in the SC-URA broth. A second-step counterselection was performed by growing the colonies from SC-LEU+5FOA plates in liquid SC-LEU+5FOA medium for 1-2 days and spreading onto SC-LEU+5FOA plates. The plasmid cure was verified by picking the colonies that grew on a SC-LEU+5FOA plate but not on a SC-URA plate.

### Citramalate Production

To compare transformants with various cimA sequences, cells were harvested from a fresh agar culture plate, and then resuspended in 50 mL SC-URA to let the starting OD₆₀₀ reach 2. After growth at 30° C. at 200 rpm for 24 h, the supernatants were centrifuged (800 g, 5 min) and filtered (0.45 μm), then analyzed for citramalate concentration using high-performance liquid chromatography (HPLC) analysis. To select top citramalate producers from the genome-integrated-cimA strain library, strains that were confirmed to have a genome-integrated version of GFP were inoculated in 10 mL SC-LEU broth and cultured for about 1 day. Cell pellets were collected by centrifugation, washed twice with water, transferred into 10 mL of SC-LEU with 50 g/L glucose liquid medium with an initial OD₆₀₀ of 1, and cultivated at 30° C. with 250 rpm orbital shaking in 55 mL glass tubes. Samples (1 mL cell culture) were collected after 5-day growth for citramalate analysis. After removal of URA plasmid from top citramalate producers, the new genome-integrated-cimA strains were cultivated under different media (SC+50 g/L glucose and YPD+50 g/L glucose) and compared with the plasmid version strain SD108 ura3Δ pCimA03 for quantifying metabolite production, using the wild-type SD108 used as a control strain. Seed cultures were grown in 10 mL YPD liquid medium and cultured for about 1.5 day. The fermentation condition was the same as for the abovementioned method except that samples (0.5 mL cell culture) were collected at 24 h, 48 h, and 72 h, and ODs were also measured. The experiments were conducted with three biological replicates.

### HPLC and LC-MS Analysis

To identify I. orientalis strains with active cimA genes, the spent media samples (500 μL) were analyzed using a Shimadzu system with refractive index detectors, using a Rezex ROA Organic Acid H⁺ column at 55° C. with 5 mM H₂SO₄ (0.5 mL min⁻¹) as the mobile phase. Citramalate was identified by comparing the retention times with commercial standards (Sigma), and concentrations were determined from calibration curves. For quantification of citramalate production after fermentation, spent media was analyzed by an Agilent 6495C liquid chromatography mass spectrometer (LC-MS), equipped with an electron spray ionization source coupled to a triple quadrupole mass analyzer. The spent media was diluted 50- to 300-fold into 40:40:20 methanol:acetonitrile:water. Chemical separation was based on hydrophilic interaction liquid chromatography (HILIC) with an XBridge BEH Amide column (2.1 mm×150 mm, 2.5 μm particle size, 130 Å pore size; Waters), with a solvent gradient as follows: 10% A at 0 min, 25% A at 3 min, 30% A at 8 min, 50% A at 10 min, 75% A at 13 min, 100% A at 16 min, 10% A at 21 min (solvent A is 20 mM ammonia and 20 mM ammonium acetate in water with 5% acetonitrile, pH 9; solvent B is 100% acetonitrile), and a flow rate of 150 μL/min. The mass spectrometer operated in a multiple reaction monitoring mode with negative ionization. The particular reactions (precursor ion->product ion) and collision energies were: glucose, 179->89, 15 V; citramalate, 147->85, 15 V; glycerol, 91->59, 15 V; pyruvate, 87->43, 12 V. For quantitation, a mixture of standards was prepared in a series of concentrations, similarly analyzed, and then used to obtain external calibration curves. Data were converted to mzXML format by msconvert (proteowizard) (Chambers et al., 2012) and analyzed by El-Maven software (Elucidata).

### Results

pH and Citramalate Tolerance of I. orientalis SD108

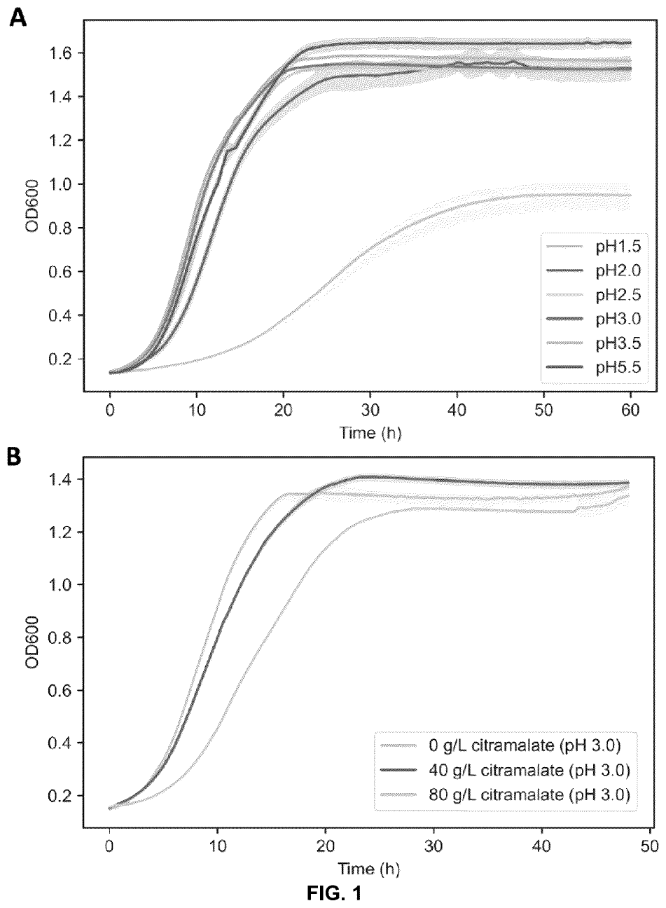

We initially tested I. orientalis's ability to tolerate low pH and high citramalate concentration to evaluate the potential of using I. orientalis as a host for citramalate production using a low-pH fermentation process. As shown in FIG. 1A, growth curves for I. orientalis SD108 were similar over a wide range, from a pH of 2.0 to a pH of 5.5. This result is consistent with results from a previous study (Xiao et al., 2014). Measuring cell density at 10 hours of cultivation, Xiao et al. concluded that optimal growth of I. orientalis SD108 occurred at a pH range of 3 and 6. To our surprise, we analyzed growth curves and found that I. orientalis SD108 can grow at a pH of as low as 1.5, although at that pH the growth rate was slower than it was under other pH values (FIG. 1A). At a pH of 1.5, I. orientalis SD108 took 40 hours to reach an OD₆₀₀ of 0.9, which was approximately 4 times longer than the time it took to reach the same density at pHs of 2.5, 3.0, 3.5, and 5.5. Additionally, we evaluated the ability of I. orientalis SD108 to tolerate citramalate at two different concentrations (40 g/L and 80 g/L). We set this culture pH at 3.0 because the pKa of citramalate is around 3.35, and we expected that the culture pH would be maintained around the pKa of citramalate. We found that I. orientalis SD108 could tolerate 80 g/L of citramalate at pH 3.0 and maintain a growth rate of about 50% of the control's (FIG. 1B). These properties of I. orientalis SD108 make it an ideal candidate as a host platform for production of citramalate through a low-pH fermentation process.

Identification of cimA for Citramalate Production in I. orientalis

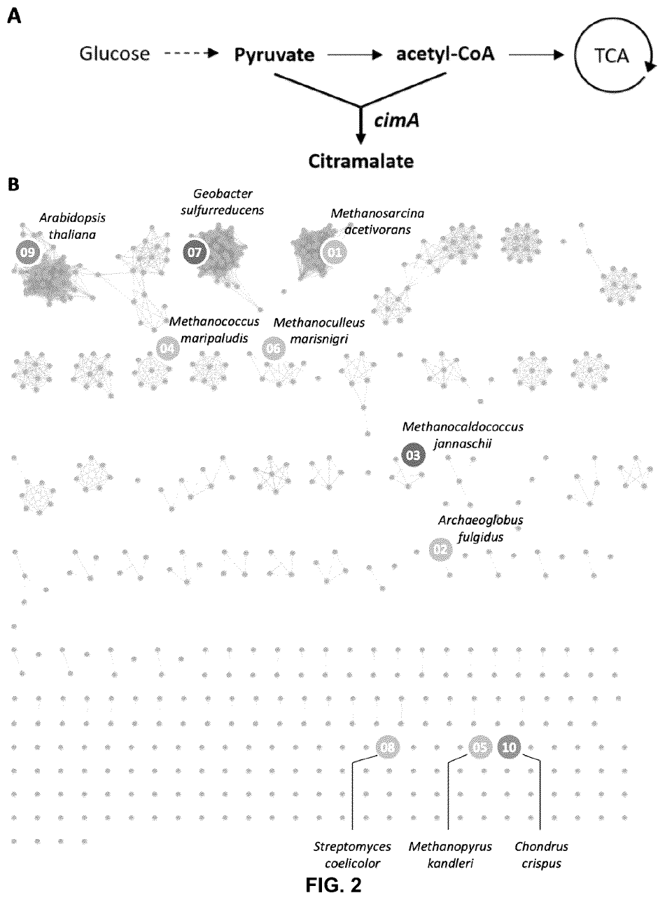

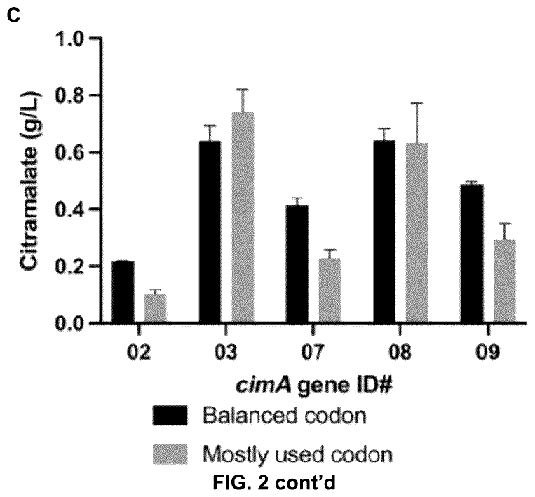

To produce citramalate efficiently, we first sought a cimA variant more compatible with expression in I. orientalis and thereby better for citramalate production (FIG. 2A). We built the SSN and selected ten cimA variants to maximize the sampling space across the SSN (FIG. 2B). We subsequently synthesized these genes, cloned them into a plasmid, expressed them in I. orientalis, and measured citramalate production. Five out of the ten genes showed citramalate synthase activity. The two strains carrying cimA gene #03 (Methanocaldococcus jannasch) (one synthesized using a “balanced” codon optimization strategy, the other using a “mostly used” strategy) averaged the highest productivity in citramalate production (0.64 g/L and 0.74 g/L, respectively) (FIG. 2C). The two strains carrying cimA gene #08 (Streptomyces coelicolor) averaged the second highest productivity in citramalate production (0.63 g/L and 0.64 g/L, respectively) (FIG. 2C). We then evaluated these two high-performing CimA variants for their activities to produce citramalate through an in vitro analysis using pyruvate and acetyl-CoA as substrates. We collected the data in technical triplicate. The calculated specific activities for Methanocaldococcus jannasch CimA and Streptomyces coelicolor CimA are 0.38 (SD=0.023) and 0.55 (SD=0.2) μmol/min/mg, respectively. The strains carrying the other three cimA genes showed much lower citramalate production, 0.1-0.4 g/L. Thus, we identified two cimA variants that have good activity. Because gene #03 (“mostly used”) produced slightly more citramalate than the others, we selected it for subsequent studies. However, the other variants could also be integrated into I. orientalis to effectively increase the copy number of cimA, relieving the concern of potential recombination among the repeats if the identical sequence were integrated for multiple times.

Transposon-mediated genome integration for citramalate production

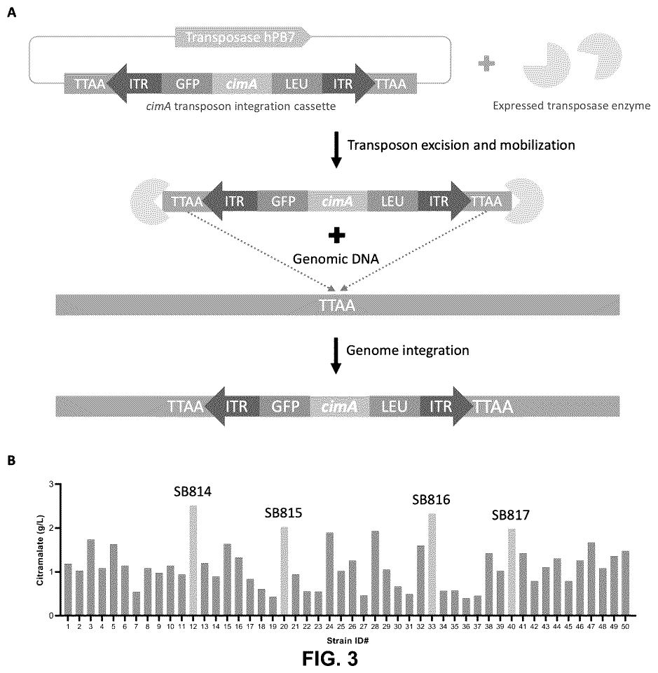

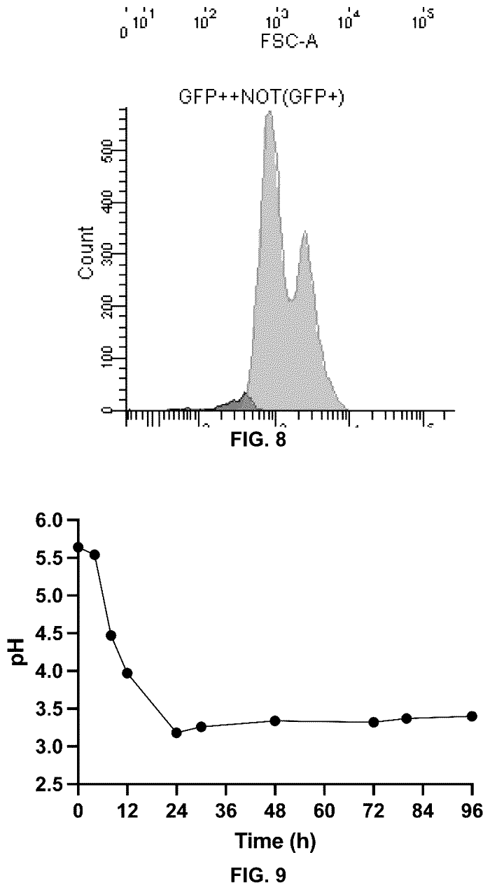

We selected the piggyBac transposon system to integrate the cimA gene into the I. orientalis genome. This system can integrate multiple copies of a payload into random locations (any TTAA sites) of the genome. In this way, we could simultaneously evaluate the effects of different integration locations and copy numbers of the cimA gene on citramalate production. A plasmid, pWS-URA-hPB7-GFP-CimA-LEU containing a hyperactive piggyBac transposase gene (hPB7) and the transposon, GFP-CimA-LEU gene cassette, flanked by inverted repeat sequences (IRSs) was constructed. This integration cassette is also flanked by extra TTAA, so we could expect the cassette to be integrated into any TTAA sites in the I. orientalis genome (FIG. 3A). After transformation of pWS-URA-hPB7-GFP-CimA-LEU, we randomly picked 50 colonies from the SC-LEU plate and determined whether the cimA transposon integration cassette was integrated using flow cytometry. All 50 strains stably expressed GFP, suggesting the successful integration of the cassette into the genome (Supplementary FIG. 8). These strains were cultured in SC-LEU media containing 50 g/L glucose for 5 days, and citramalate production was measured. The lowest and highest citramalate production differed 6-fold, ranging from 0.4 to 2.5 g/L (FIG. 3B). We subsequently selected the top four producers and counter-selected to cure the plasmids and to ensure stable cimA expression. For simplicity, these four strains, #12, #20, #33, and #40, were renamed SB814, SB815, SB816, and SB817, respectively.

To determine the integration locations and copy number of cimA, we performed PacBio sequencing and summarized the results in Table 1 and Table 6. We identified the copy number of cimA and the integration sites by aligning raw reads to the genome sequence of I. orientalis SD108 v2.0 from the JGI MycoCosm, The Fungal Genome Resource database (Grigoriev et al., 2014). We also identified the neighborhood genes of each cimA integration site based on the data retrieved from the JGI IMG Integrated Microbial Genomes and Microbiomes database (Chen et al., 2019). The strain SB814 had the most cimA copies in the genome (six). Two of the six copies disrupted a hypothetical protein gene (these two loci are allelic to each other). Strains SB815, SB816, and SB817 each had two cimA copies. The cimA in SB815 did not integrate into any known gene, while the cimA integration site of SB816 disrupted a myosin protein heavy chain (MHC) gene. The cimA in SB817 destroyed a AAA family ATPase gene and its allele, and its transposase recognition was CTAA, not TTAA. These four genome-integrated-cimA strains were cultured, and their ability to produce citramalate was further evaluated.

TABLE 1
Copy number and integration sites
of cimA-integrated I. orientalis strains.

| | | | | Gene |
|---|---|---|---|---|
| | | Trans- | | that has |
| I. | Copy | posase | | been |
| orientalis | num- | recog- | | dis- |
| strain | ber | nition | Integration site/allele site^{a} | rupted^{b} |
| SB814 | 6 | TTAA | Issorie2\|scaffold_1: 422225- | None |
| | | | 42228/scaffold_13: | |
| | | | 305227-305230 | |
| | | TTAA | Issorie2\|scaffold_29: 112489- | Hypo- |
| | | | 112492/scaffold_ | thetical |
| | | | 31: 102246-102249 | protein |
| | | TTAA | Issorie2\|scaffold_10: 140124- | None |
| | | | 140138/scaffold_1: 1572178- | |
| | | | 1572193 | |
| SB815 | 2 | TTAA | Issorie2\|scaffold_1: 2431243- | None |
| | | | 2431246/ | |
| | | | scaffold_20: 160384-160387 | |
| SB816 | 2 | TTAA | Issorie2\|scaffold_2: 718184-718187 | None |
| | | TTAA | Issorie2\|scaffold_34: | Myosin |
| | | | 30875-30987 | protein |
| | | | | heavy |
| | | | | chain |
| SB817 | 2 | CTAA | Issorie2\|scaffold_53: 4781-4784/ | AAA |
| | | | scaffold_1: 72387-72390 | family |
| | | | | ATPase |

^{a}The integration sites were identified based on the JGI MycoCosm, The Fungal Genome Resource.
^{b}The gene neighborhood data was retrieved from the JGI IMG Integrated Microbial Genome & Microbiomes database.

Production of Citramalate from cimA-Integrated I. orientalis Strains

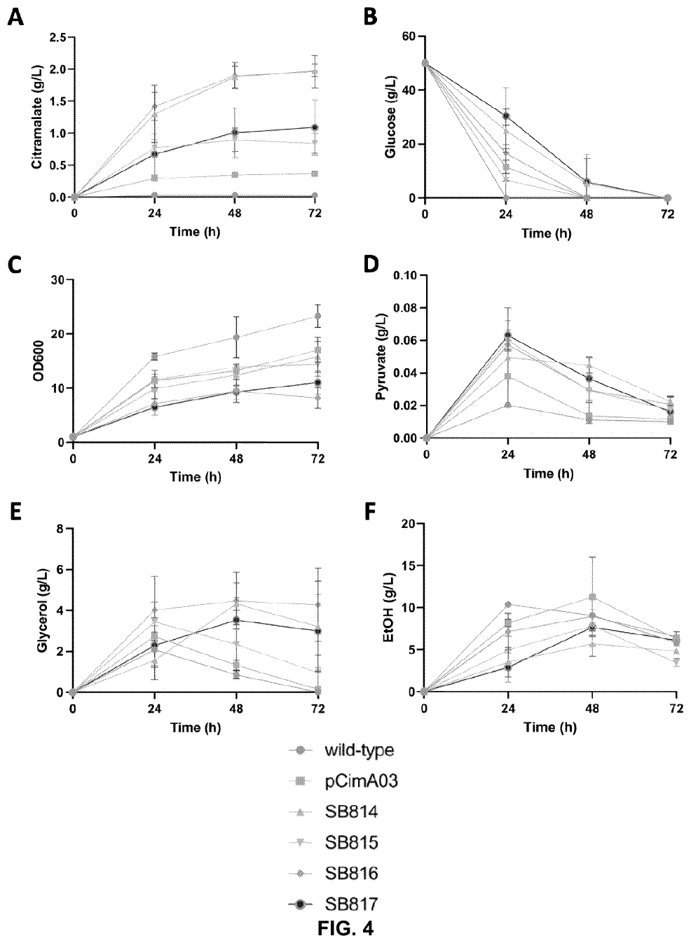

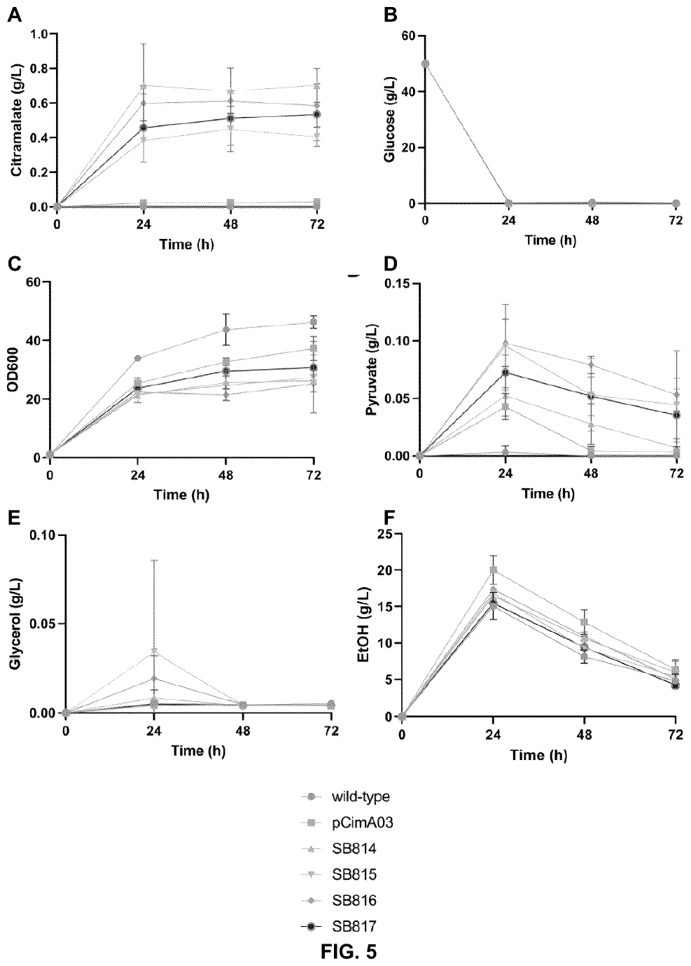

To compare the performance of different strains, we cultured the I. orientalis SD108 ΔURA3 strain, that same strain but harboring the cimA plasmid (pCimA03), and the top four citramalate producers (SB814, SB815, SB816, and SB817) with the genome-integrated-cimA in both SC and YPD media containing 50 g/L glucose for 3 days. In the SC medium, glucose consumption and growth rate were reduced for all engineered strains (FIG. 4). They did all produce citramalate but in different amounts. The four genome-integrated strains all produced significantly more citramalate than their plasmid-based counterpart did (FIG. 4A and FIG. 5A). In particular, SB814 and SB816 produced the most citramalate, with a titer of 2.0 g/L and a yield of up to 7% mol citramalate/mol consumed glucose (FIG. 4A). In contrast, all strains cultured in the YPD medium consumed 50 g/L glucose within 24 h (FIG. 5B). Interestingly, cultures in the YPD medium did not increase citramalate production as much as cultures in the SC medium did, but the YPD medium doubled biomass and ethanol production (FIGS. 5C and 5F). Supplementary FIG. 9 shows the pH values of the YPD medium containing SB814 over 96 hours. The results support the viability of using I. orientalis as a host platform for producing citramalate under low-pH fermentation conditions, as the pH was observed to rapidly drop to 3.2 within 24 hours and remain stable between 3.2 and 3.4 until the end of the cultivation period.

### Discussion

Identification of Optimal CimA Variants for Citramalate Production in I. orientalis

Since citramalate synthase (CimA, EC 2.3.1.182) was identified from a thermophilic methanogenic archaea, M. jannaschii (Howell et al., 1999), only a few CimA variants have been evaluated in an E. coli heterologous expression system for citramalate biosynthesis (Webb et al., 2018; Wu and Eiteman, 2016). With the rapid increase in the abundance of protein sequences in public databases, we sought to identify more efficient CimA from nature. We therefore built a CimA SSN to investigate this possibility and guide target gene selection. In our SSN (FIG. 2B), sequences sharing over 80% identities were grouped into the same cluster. By selecting candidate genes from different clusters, we were able to avoid synthesizing genes with high similarities. In this way, we identified five cimA genes that are active in I. orientalis. Their origins are Archaeoglobus fulgidus (gene #02), M. jannaschii (gene #03), Geobacter sulfurreducens (gene #07), S. coelicolor (gene #08), and Arabidopsis thaliana (gene #09). Among them, the cimA genes from M. jannaschii and G. sulfurreducens were previously functionally expressed in E. coli. Whereas the other three were verified in I. orientalis for the first time. We did not identify CimA variants that resulted in higher citramalate yields than that from M. jannaschii. Although it has only 31.2% sequence identity to CimA from M. jannaschii, CimA from S. coelicolor enabled to the production of a similar level of citramalate (FIG. 2C). Table 7 shows the sequence identity among different CimA genes. These CimA variants generally show only 30-50% identities at the protein level. This result shows that the sequence diversity of CimA is high; no obvious trend for sequence-function relationships was found.

Another factor that impacts citramalate production is the level of functional CimA expression. Codon optimization is a common strategy to increase the expression level of proteins (Plotkin and Kudla, 2011). We therefore used two different codon optimization parameters offered by the JGI BOOST; one is “balanced” and the other is “mostly used.” In “balanced” codon optimization, BOOST selects the most-used and second-most-used codon for each amino acid as evenly used as possible during the process (Oberortner et al., 2017). This mitigates the sequence complexity that may arise by using only the most-preferred codon, as is done when using the “mostly used” optimization strategy. Since low-complexity DNA reduces the occurrence of repeats, secondary structure, and sequence stretches with extreme GC content, we expected DNA to be readily manufactured, and could potentially avoid mRNA secondary structure that might affect protein expression. Although we did not evaluate the CimA protein level in this study, the “balanced” codon-optimized cimA genes generally produced more citramalate (FIG. 3C). This difference between “balanced” and “mostly used” in the plasmid expression system is subtle. Access to multiple cimA variations is helpful because eventually, numerous copies of cimA genes need to be integrated into the genome to enhance the production level of citramalate, and the potential instability factor such as recombination among identical sequences needs to be mitigated.

cimA Genome Integration by piggyBac Transposase System

Plasmid expression systems are typically unstable and not favorable for metabolic engineering. In contrast, genome integration is a better approach to stably maintain heterologous genes. The gene numbers and integration locations are also known to be crucial for heterologous gene expression (Da Silva and Srikrishnan, 2012; Flagfeldt et al., 2009). Our top producer, SB814, has the most (six) cimA copies in the genome. Compared with the other three strains, which have only two copies (SB815, SB816, and SB817), SB814 had the highest citramalate production in both SC and YPD medium (FIG. 4A and FIG. 5A). However, one of the strains with only two cimA copies, SB816, had a production level similar to that of SB814 in the SC medium (FIG. 4A), and had only a slightly lower production level than SB814 in the YPD medium (FIG. 5A). This may be because the integration site in SB816 allows a high-level gene expression or expression dynamics more suitable for citramalate production. Although the difference in gene expression level requires further verification, the result suggests the potential of these integration loci providing more choices for future strain engineering. In general, we only recommend using GFP expression and flow cytometry to facilitate the screening of integrants rather than as a guide to report the production of a target compound. This is because a high titer is a result from collective factors such as enzyme concentration and activity, substrate availability, depletion of important central metabolites, toxicity of intermediates and products, and key enzyme expression dynamics. In our previous study with optimization of shikimate production in a different yeast host, the correlation between GFP expression and high production was also not observed (Zhao et al. 2020).

Considering random integration could accidentally disrupt coding regions, the fatality of the disruption needs to be examined. In our case, although the integration sites in three cimA-integrated I. orientalis strains disrupted genes (Table 1), including a hypothetical protein gene in SB814, a myosin protein heavy chain (MHC) gene in SB816, and an AAA family ATPase gene in SB817, the strains' growth showed that no fatal effects had occurred. Their growth rates were also not dramatically different than that of SB815, which did not have any genes disrupted (FIG. 4C and FIG. 5C). The MHC gene was labeled non-essential for cell survival under laboratory growth conditions in S. cerevisiae (Rodriguez and Paterson, 1990). The AAA family ATPase contains many genes with similar functions. According to the annotation data of Pfam (PF00004) in the JGI's MycoCosm database, I. orientalis has 32 AAA family ATPases (Grigoriev et al., 2014). The disrupted gene in SB817 was likely compensated by other ATPases, so no fatal effect occurred. Although the growth rates of all four cimA-integrated I. orientalis strains were slower than that of the wild-type and the strain expressing cimA in a plasmid, those four strains still reached the late logarithmic phase at 24 hours. The slower growth might also have been caused by the extra expenditure for producing a foreign product or LEU2 auxotroph.

Recently, a study describes a Hermes transposon-mediated random integration method in Scheffersomyces stipitis by transforming a nonreplicable circular DNA allows the skip of the plasmid curing step and efficiently removes false positive clones (Zhao et al., 2020). We transformed a nonreplicable circular DNA containing the ITR flanked GFP-CimA-LEU fragment and PiggyBac cassette but were only able to get very few colonies on the plate. The number of colonies was too few for effective library construction and screening. Previous studies have shown that Nonhomologous-End-Joining (NHEJ) is involved in the double-stranded DNA break repair in transposition (Yant and Kay, 2003; Yu et al., 2004). However, I. orientalis is a homologous recombination-dominant strain (Cao et al., 2020), and the transient expression of PiggyBac in the nonreplicable carrier may not be sufficient for transposition. Future studies to identify important NHEJ-related proteins and overexpress these proteins may help streamline the protocol via a nonreplicable circular DNA in I. orientalis.

Citramalate Production from cimA-Integrated I. orientalis Strains and Future Optimization

The much higher production yielded by the cimA-integrated strains in general than the one expressing cimA in a plasmid and distinctly different levels of citramalate production among the four integration strains (FIG. 4A and FIG. 5A) support the validity of using the piggyBac transposase system to integrate the heterologous gene directly into the I. orientalis genome and exploring the impacts of integration loci and copy numbers on citramalate production.

In general, we observed higher citramalate production from cultures using the SC medium (FIG. 4A) than from those using the YPD medium (FIG. 5A). In the YPD medium, cells quickly consumed glucose and mainly diverted the carbon to their growth and to ethanol production (FIGS. 5C and 5F). In both medium conditions, post glucose depletion, the strains shifted their metabolism to consume ethanol for growth (FIG. 4F and FIG. 5F). However, ethanol consumption did not lead to further citramalate production.

Deletion of pyruvate decarboxylase (PDC) and/or downregulation of the TCA cycle have been shown to reduce efflux to ethanol synthesis (Webb et al., 2018; Wu and Eiteman, 2016; Xiao et al., 2014). However, it is known that the deletion of PDC will negatively affect the cytosolic synthesis of acetyl-CoA. To increase cytosolic acetyl-CoA level, expression of pyruvate dehydrogenase (Nielsen, 2014), and/or non-oxidative glycolysis (NOG) pathways (Meadows et al., 2016) may be considered. Alternatively, it is also conceivable to express CimA in the mitochondria, where both pyruvate and acetyl-CoA are accessible through pyruvate dehydrogenase activity.

The engineered strains, in particular the cimA-integrated strains, accumulated glycerol more than the wild-type strain in the SC medium (FIG. 4E), suggesting that the expression of citramalate synthase may also cause metabolic imbalance (Vemuri et al., 2007). Conversion of glucose into citramalate yields excess reducing co-factors, which might be offset by the production of glycerol. Production of acetyl-CoA through pyruvate oxidase and the NOG pathway may also mitigate the redox imbalance caused by citramalate production and help increase citramalate yield. Combining the above strategies to further increase the yield of citramalate should be considered for future strain engineering.

### CONCLUSION

Bio-based organic acids are important chemical building blocks for the production of commodity chemicals and materials with diverse applications. The non-conventional chassis I. orientalis has an extraordinary ability to tolerate diverse industrially relevant stresses (e.g., low pH and inhibitors in lignocellulosic biomass hydrolysates), and it as a chassis for the production of organic acids could potentially reduce the cost and environmental footprint of organic acid production by 30% compared with using conventional species as chassis. For non-model strains, genetic engineering tools are limited, and the Design-Build-Test-Learn cycle tends to be slow. Therefore, we decided to use the piggyBac transposon system to identify optimal integration loci and copy numbers for citramalate production. We used the M. jannaschii cimA, which performed the best in I. orientalis according to our initial screening. Four strains, SB814 through SB817, showed high citramalate production after random integration of this cimA gene using the piggyBac system. Further characterization indicated that these strains contain 2 to 6 copies of the cimA gene in their genomes, and their integration sites were diverse. We demonstrated that SB814 and SB816 produced the highest amount of citramalate, 2 g/L, which was 6-fold higher than that of their plasmid counterpart. These results demonstrated the efficacy of the piggyBac transposon system for rapid exploration of integration sites and copy numbers of important metabolic genes, which allowed us to create high-production strains.

TABLE 2
Strains and plasmids used in this study.

| Strain/plasmid | Features | Source |
|---|---|---|
| Strains | | |
| E. coli TOP10 | Cloning host | Invitrogen ™ |
| E. coli | lacI^{q} rrnB_{T14} ΔlacZ_{WJ16} ΔphoBR580 | (Shao et al., |
| BW25141 | hsdR514 ΔaraBAD_{AH33} ΔrhaBAD_{LD78} | 2009) |
| | galU95 endA_{BT333} uidA(ΔmluI)::pir+ | |
| | recA1, cloning host | |
| S. cerevisiae | MATα, ade2-1, ade3422, ura3-1, | (Shao et al., |
| YSG50 | his3-11, 15, trp1-1, leu2-3, 112, | 2009) |
| | and can1-100, cloning host | |
| I. orientalis | Wild-type | (Xiao et al., |
| SD108 | | 2014) |
| I. orientalis | ura3Δ, host for cimA expression | (Xiao et al., |
| SD108 | | 2014) |
| I. orientalis | ura3Δleu2Δ, host for cimA genome | (Xiao et al., |
| SD108 | integration | 2014) |
| Plasmids | | |
| pZF_TDH3p | Expression vector backbone | (Xiao et al., |
| | | 2014) |
| pZF_EcoR1_ | cimA expression vector | This study |
| TDH3p | | |
| pScARS/CEN-L | Transposase plasmid backbone | (Cao et al., |
| | | 2020) |
| pWS-URA-hPB7- | Transposase plasmid | This study |
| GFP-CimA-LEU | | |

TABLE 3
Summary of CimA sequence similarity network.

| Query sequence | UniProt ID Q58787 |
|---|---|
| Database version | UniProt: 2019 August/InterPro: 76 |
| Number of sequences | 636 (UniProt ID matches) |
| Number of unique sequences | 576 |
| Alignment score | 250 |
| Sequence percent identity for | 80% |
| clustering | |
| Total number of edges | 201,930 |
| Convergence ratio | 1.000 |

TABLE 4
DNA sequence of cimA with “balanced” codon usage.

| ID | Sequence | Description |
|---|---|---|
| 01 | ATGAGAGATGGTGAACAGACTCCGGGAGTTGCTTTAACAAGGGAAAAGA | From |
| | AGCTACTCATCGCGCGTGCATTAGATGAGATGAGAATTAATGTCATCGA | Methanosarcina |
| | AGCCGGGTCTGCTATTACCAGTGCCGGAGAAAGAGAGTCCATTAAGGCA | acetivorans |
| | GTTGCTAATGCTGGATTAGACGCAGAAATCTGTAGTTATTGTAGAATTG | |
| | TGAAGATGGATGTGGATCATGCCCTCGAGTGTGATGTTGATTCAATTCA | |
| | TTTGGTAGCTCCAGTGAGTGACCTCCACATTAAAACCAAGATCAAGAAG | |
| | GATAGAGATACTGTTAGACAGATCGCCGCAGAGGTCACAGAGTACGCAA | |
| | AGGATCATGGTTTAATCGTTGAACTATCCGGCGAGGACGCCTCGAGAGC | |
| | CGATCCAGAATTTTTAAAGGCAATTTACTCTGACGGTATTGACGCGGGA | |
| | GCTGACAGATTGTGCTTTTGCGATACCGTCGGTCTATTGGTTCCAGAGA | |
| | AAACAACTGAGATCTTTCGTGACCTTTCCAGTTCCTTGAAGGCACCTAT | |
| | TTCTATTCATTGTCACAACGACTTCGGCCTTGCTACAGCCAACACAGTC | |
| | GCTGCATTAGCTGCTGGTGCAAAGCAGTCCCATGTGACAATTAACGGAC | |
| | TTGGTGAAAGAGCTGGTAATGCCTCGTTGGAAGAAGTTGTCATGTCGCT | |
| | CGAGTGGTTATACAAGTACGACACTGGAATCAAACATGAGCAGATCTAT | |
| | AGAACATCAAGATTGGTTTCGAGATTAACAGGTATTCCCGTGAGTCCAA | |
| | ATAAGGCATTGGTTGGTGGTAATGCTTTCACTCACGAAGCAGGAATCCA | |
| | TGTCCACGGCTTGTTAGCGGATAAGTCGACCTATGAACCTATGTCGCCA | |
| | GAGTACATCGGTAGACAAAGACAAATCGTGCTTGGCAAGCACGCGGGTC | |
| | GTTCTTCTATTACTTTGGCATTGAAGGAAATGGGTTTGGAGGCCGATGA | |
| | AGCTCAAACTGAAGAAATCTTTAACAGAGTTAAGCAAATGGGTGACCAG | |
| | GGTAAGCATATTACTGATGCAGATTTGCAAACCATTGCTGAAACAGTCT | |
| | TAGACATTTATAAGGAGCCTATCGTGAAATTAGAAGAATTTACAATTGT | |
| | CTCCGGAAATCGTGTCACCCCTACCGCCTCTATTAAATTGAATGTGAAA | |
| | GACAAGGAGATCGTCCAGGCCGGTATCGGTAATGGTCCTGTTGATGCAG | |
| | TGATTAACGCTATTCGTAGAGCCGTGAGTTCTTGCGCCGAGGATGTTGT | |
| | TCTTGAAGAATACCATGTTGATTCCATAACTGGTGGTACGGATGCACTT | |
| | GTTGAAGTGAGAGTGAAGCTATCAAAAAACGGTAAGGTTATCACAGCTT | |
| | CAGGTGCTAGAACTGATATAATCATGGCCTCAGTTGAAGCAGTCATGAA | |
| | TGGTATGAACAGGTTAATTCGTGAAGAATAA (SEQ ID NO: 1) | |
| 02* | ATGCAAGTCAAGATCCTTGATACAACATTGCGTGACGGTGAGCAAACCC | From |
| | CTGGTGTTTCTTTGTCCGTCGAACAAAAGGTGATGATCGCAGAAGCTCT | Archaeoglobus |
| | CGACAACCTTGGAGTTGATATTATCGAAGCGGGTACTGCCATAGCCTCT | fulgidus |
| | GAAGGGGATTTTCAAGCAATTAAGGAAATTTCACAAAGAGGTCTCAATG | |
| | CCGAAATCTGTTCATTCGCGAGAATTAAGCGAGAGGACATCGACGCTGC | |
| | CGCCGATGCTGGTGCAGAGTCAATCTTTATGGTGGCTCCATCGTCTGAT | |
| | ATTCATATAAACGCCAAGTTTCCAGGAAAGGACAGAGACTACGTCATCG | |
| | AGAAATCAGTCGAAGCTATTGAATATGCAAAGGAAAGAGGCCTCATTGT | |
| | GGAATTCGGTGCTGAAGATGCATCAAGAGCCGACCTCGATTTCGTTATT | |
| | CAATTGTTCAAAAGAGCGGAGGAGGCAAAGGCCGATAGAATCACATTCG | |
| | CGGACACTGTTGGAGTGCTTTCTCCTGAAAAGATGGAAGAAATTGTGAG | |
| | AAAGATCAAAGCAAAGGTTAAATTGCCATTAGCTATACACTGTCATGAC | |
| | GATTTCGGCTTGGCAACCGCTAACACTATTTTTGGTATTAAGGCCGGCG | |
| | CGGAAGAATTTCATGGCACGATTAACGGTTTGGGTGAGAGGGCAGGCAA | |
| | TGCCGCCATCGAAGAGGTTGTTATCGCATTGGAATACCTTTACGGTATT | |
| | AAGACCAAAATTAAGAAGGAAAGATTGTACAATACTTCTAAGCTCGTGG | |
| | AGAAGTTGTCCCGTGTCGTCGTTCCACCAAACAAGCCAATTGTCGGAGA | |
| | TAACGCTTTCACTCATGAGTCCGGTATCCATACTTCTGCATTGTTCAGA | |
| | GATGCAAAATCCTACGAGCCCATCTCGCCTGAAGTTGTTGGTAGGAAGA | |
| | GGGTCATCGTTTTGGGTAAGCACGCTGGTAGGGCAAGCGTTGAAGCAAT | |
| | TATGAATGAATTAGGTTACAAGGCTACCCCGGAACAGATGAAGGAAATT | |
| | CTAGCTAGAATTAAGGAAATTGGTGATAAGGGTAAAAGAGTTACCGATG | |
| | CTGATGTTCGAACAATAATTGAAACTGTGTTGCAAATTAAAAGAGAAAA | |
| | AAAAGTCAAGCTTGAGGATTTAGCAATCTTCTCTGGTAAGAACGTCATG | |
| | CCCATGGCGTCAGTCAAGTTGAAAATTGACGGTCAAGAGAGAATTGAGG | |
| | CCGCTGTTGGATTAGGACCAGTCGATGCCGCAATTAACGCAATCAGGAG | |
| | AGCAATTAAAGAATTTGCGGATATCAAATTAGTTTCCTACCATGTTGAC | |
| | GCCATTACAGGAGGTACGGACGCCCTCGTTGATGTCGTTGTTCAGTTGA | |
| | AGAAAGACAACAAGATTGTTACGGCACGTGGTGCGAGGACAGATATTAT | |
| | TATGGCATCCGTTGAAGCATTCATCGAGGGTATTAATATGCTCTTCTAA | |
| | (SEQ ID NO: 2) | |
| 03* | ATGATGGTTAGGATTTTCGACACTACTTTAAGAGATGGTGAACAGACTC | From |
| | CAGGTGTTTCTCTGACCCCAAACGATAAGTTGGAAATCGCAAAGAAATT | Methano- |
| | GGATGAATTGGGGGTTGATGTTATTGAGGCAGGATCAGCTATTACTTCT | caldococcus |
| | AAGGGAGAAAGAGAAGGTATCAAGCTCATTACTAAGGAGGGTTTGAACG | jannaschii |
| | CAGAAATCTGTTCCTTCGTCCGTGCATTGCCTGTCGATATCGATGCGGC | |
| | GTTAGAGTGTGATGTCGATTCGGTTCATTTAGTGGTGCCTACATCCCCA | |
| | ATCCATATGAAGTACAAATTGAGGAAGACGGAGGATGAAGTCTTGGAAA | |
| | CGGCGTTGAAGGCAGTCGAATATGCTAAGGAGCATGGTTTAATAGTCGA | |
| | GTTGTCGGCGGAAGATGCGACCAGATCCGATGTTAACTTCTTGATCAAG | |
| | TTGTTCAATGAAGGTGAAAAGGTCGGTGCAGATAGAGTCTGTGTTTGTG | |
| | ATACCGTTGGTGTCCTTACACCTCAAAAGTCACAAGAATTGTTTAAGAA | |
| | GATTACTGAAAATGTTAACCTCCCCGTTTCAGTCCATTGCCATAACGAT | |
| | TTTGGTATGGCGACAGCTAATACATGTTCTGCGGTCTTGGGTGGCGCTG | |
| | TCCAGTGTCATGTCACAGTTAACGGAATTGGTGAAAGAGCAGGAAATGC | |
| | CTCATTGGAGGAAGTTGTTGCCGCATTAAAAATTTTGTACGGTTATGAT | |
| | ACTAAGATTAAAATGGAAAAGTTGTACGAAGTGTCCCGTATCGTTTCGA | |
| | GATTAATGAAGTTGCCTGTGCCTCCAAACAAAGCCATCGTGGGCGATAA | |
| | TGCTTTCGCCCATGAAGCCGGTATTCATGTTGATGGTCTCATTAAAAAC | |
| | ACTGAAACGTATGAGCCTATCAAGCCAGAGATGGTTGGTAACCGTCGTA | |
| | GAATTATTTTGGGTAAACATTCTGGTAGAAAAGCACTCAAATATAAGTT | |
| | AGACTTGATGGGAATTAACGTTTCTGATGAACAGTTGAATAAGATTTAT | |
| | GAGCGTGTCAAGGAGTTCGGTGACTTGGGAAAGTATATTTCCGATGCAG | |
| | ATTTACTGGCAATTGTGAGAGAAGTTACGGGTAAGTTAGTTGAGGAAAA | |
| | GATTAAGTTGGACGAGTTGACCGTTGTTTCGGGGAATAAGATTACTCCA | |
| | ATTGCCTCCGTTAAGCTCCATTACAAGGGAGAGGATATTACTTTGATTG | |
| | AAACAGCCTATGGCGTTGGACCAGTGGACGCGGCGATCAATGCAGTCAG | |
| | AAAGGCAATTTCGGGTGTTGCTGACATCAAGTTAGTTGAGTACAGGGTT | |
| | GAGGCCATCGGGGGTGGTACTGATGCATTGATCGAAGTCGTTGTCAAGC | |
| | TTAGAAAGGGCACTGAAATAGTCGAAGTGAGGAAGTCCGATGCCGACAT | |
| | CATTAGAGCTTCAGTCGACGCAGTGATGGAAGGTATCAACATGTTACTT | |
| | AATTAG (SEQ ID NO: 3) | |
| 04 | ATGATTGTCTTGTTTGTTGAACCCATTAGGTTCTTTGATACCACATTAA | From |
| | GAGATGGTGAGCAAACTCCAGGCGTTAGTTTGACACCTGCCGGAAAGCT | Methanoculleus |
| | GGAAATTGCAACACACTTAGCCGATGTTGGGGTCCATGTTATCGAAGCA | marisnigri |
| | GGTTCCGCAGCGGCTTCTGTTGGAGAACGTGAGTCCATTAGAGCGATTG | |
| | CAGACGCAGGTTTAGCAGCCGAGTGTTGTACCTACGTCAGGGCATTACC | |
| | AGGCGATATTGATTTAGCTGCCGATGCGGGCGCCGATTCTGTCCACTTG | |
| | GTCGTTCCTGTCTCTGATTTGCACATTGCTAAGAAGTTGAGGAAGACTA | |
| | GAGAACAGGTTTCTGAGATGGCCTGGTCCGCAGTTGAATATGCCAAGGA | |
| | AAGAGGTTTGGTTGTTGAGTTGTCAGGTGAAGATGCGTCGAGAGCAGAT | |
| | CAGGATTTTTTGGCAGAAGTTTTTAGAGAAGGCGTTGAAAGAGGTGCTG | |
| | ATCGATTATGTTTTTGTGATACCGTCGGTTTACTGACACCAGAAAGAGC | |
| | CGCCGCAATTATTCCACCTCTTCTTTTCGCGCCTTTATCGATCCACTGT | |
| | CATGATGATTTGGGGTTTGGTTTAGCAACCACAGTCGCAGCCTTGAGGG | |
| | CTGGTGCTACTTGCGCACATGTTACAGTTAATGGTTTAGGCGAACGTGC | |
| | AGGTAACACTTCGTTAGAAGAATTGGTCATGGCATTGGAAGTTCTTTAT | |
| | GGCGTCGATACGGGTATTGCCACTGAAGAATTGTATCCATTAAGTACTC | |
| | ACGTCGCAAGACTCACAGGTGTCCCATTGGCTACCAATAAGCCTATTGT | |
| | TGGCGAAATGGCGTTCACTCATGAGTCAGGAATCCACGCTCATGGTGTT | |
| | ATGCGGGACGCATCCACGTATGAACCCTTGCAACCTGAGAGAGTAGGTA | |
| | GAAGAAGAAGAATCGTTTTAGGTAAGCACTCTGGTTCAGCCGCCGTTGA | |
| | AGCTGCTTTGCATGATATGGGTTATGCACCATCGGCCGCTCAACTCAAG | |
| | GAAATTGTCGATAGAATTAAAAGACTTGGTGATGCAGGTATGAGAATCA | |
| | CCGACGCAGATATTATGGCAATTGCTGATACAGTCATGGAAATCGAATT | |
| | TACACCGTGTATCGAACTGAGGCAATTCACAATCGTTTCAGGATCTAAC | |
| | GCAATCCCAACTGCTTCGGTCACCATGCTAGTGAGAGGTGAAGAAATCA | |
| | CGGGTGCAGCCGTCGGTACAGGTCCAGTTGACGCAGCAATTAGAGCTTT | |
| | ACAAAGATCCGTTGCTGATGTTGGTTCTGTCAGATTAGATGAGTACTCG | |
| | GTTGATGCCATCACCGGTGGTACAGATGCCTTGGTGGATGTCTCCGTTA | |
| | AGTTATCTAAAGACGGTAAGACCGTTACTAGTAGAGGTGCCAGAACTGA | |
| | CATTATCATGGCATCTGTTGAAGCTGTTATTGCAGGTATGAACAGGCTT | |
| | CTCAGAGAAGAACACGAAGATAGATCGCAAGATTCCGATTAA (SEQ | |
| | ID NO: 4) | |
| 05 | ATGAGAGAAGCTAACGCAGATGCAGACCCACCAGATGAGGTTCGGATCT | From |
| | TTGATACTACTTTACGTGATGGCGAACAAACTCCTGGCGTTGCGTTAAC | Methanopyrus |
| | ACCTGAAGAAAAACTTAGAATCGCTAGAAAATTGGATGAAATTGGTGTT | kandleri |
| | GATACCATAGAAGCAGGTTTCGCAGCGGCTTCGGAAGGAGAATTGAAAG | |
| | CAATTAGAAGAATCGCAAGAGAAGAATTGGACGCGGAGGTTTGTTCGAT | |
| | GGCGAGAATGGTCAAGGGAGATGTTGATGCAGCCGTGGAGGCGGAAGCC | |
| | GATGCCGTCCACATAGTCGTTCCAACTTCAGAGGTTCATGTTAAGAAGA | |
| | AGTTAAGAATGGATAGGGAAGAGGTCTTGGAAAGAGCCAGAGAGGTCGT | |
| | TGAATATGCTAGAGATCATGGTTTGACCGTTGAAATTTCAACTGAAGAT | |
| | GGTACTAGAACAGAATTAGAATATTTGTATGAGGTGTTTGATGCATGCT | |
| | TAGAGGCTGGAGCTGAAAGGTTGGGTTACAACGATACCGTCGGTGTCAT | |
| | GGCACCTGAAGGTATGTTCTTGGCAGTCAAGAAATTACGTGAGAGAGTC | |
| | GGTGAAGACGTTATCCTCTCAGTTCACTGTCACGATGACTTTGGTATGG | |
| | CAACTGCTAATACGGTGGCAGCAGTTAGGGCAGGTGCTAGACAAGTTCA | |
| | TGTTACAGTTAATGGTATTGGTGAAAGAGCTGGTAACGCGGCATTAGAA | |
| | GAAGTTGTCGTCGTTTTGGAAGAGTTATACGGTGTGGATACTGGAATCC | |
| | GTACTGAAAGATTGACCGAGCTCTCTAAATTGGTCGAAAGATTGACTGG | |
| | CGTCAGAGTTCCCCCAAACAAGGCCGTCGTTGGTGAAAACGCCTTTACA | |
| | CACGAATCCGGTATTCATGCCGATGGTATTTTAAAGGATGAATCTACAT | |
| | ACGAACCCATCCCTCCCGAGAAGGTCGGTCATGAAAGACGTTTCGTCTT | |
| | GGGTAAGCATGTTGGTACCTCAGTCATTAGGAAGAAGTTGAAGCAGATG | |
| | GGGGTTGACGTCGACGATGAACAGTTGCTTGAAATCTTGCGTCGTCTTA | |
| | AAAGATTGGGTGATCGTGGTAAAAGAATTACAGAGGCAGATCTCAGAGC | |
| | TATTGCAGAGGATGTTCTAGGTAGACCAGCAGAGAGAGACATCGAAGTT | |
| | GAAGATTTCACAACAGTGACTGGAAAGCGTACAATTCCAACTGCGTCGA | |
| | TTGTTGTCAAAATTGATGGTACACGTAAAGAAGCTGCTTCAACCGGTGT | |
| | CGGTCCAGTCGATGCAACTATCAAGGCTTTAGAGCGTGCATTGAAGGAT | |
| | CAGGGTATTGATTTCGAACTGGTTGAGTATCGAGCAGAAGCATTGACTG | |
| | GAGGTACCGATGCCATTACGCATGTTGACGTCAAGTTGAGGGATCCTGA | |
| | AACTGGTGATATTGTCCACTCAGGTTCGTCGAGAGAAGATATTGTCGTT | |
| | GCTAGTCTTGAGGCCTTCATTGATGGTATTAACTCTTTGATGGCAAGAA | |
| | AGAGATCTTGA (SEQ ID NO: 5) | |
| 06 | ATGGAATCCTACTTGCACTCTAACGAGATCATCAAGAATTCCTTAAAGT | From |
| | CTATGAAATTGCCCAAAAAGGTTAGAGTTTTCGATACTACACTCCGGGA | Methanococcus |
| | CGGTGAACAAACTCCTGGTGTCTCCTTGACCCCAGATCAGAAGCTAGAC | maripaludis |
| | ATTGCCACCAAGTTGTCAGAAATTGGTGTTGATGCAATTGAGGCAGGTT | |
| | TCCCTGTTTCCAGCGAGGGTGAACAAGAATCGATCAAGAAGATTACCTC | |
| | TATGGGTTTGAACGCTGAAATTTGCGGTCTTGCTAGAGCTGTGAAGAAG | |
| | GATATCGATATTGCCATCGACTGTGGTGTCGATTCCATTCATACTTTTA | |
| | TTGCAACTTCTCCTTTACATAGGGAGTATAAGCTCAAGATGTCTAAGGA | |
| | GAAGATCATTGATATCGCAATTGAATCCATTGAATACATCAAGGAGCAC | |
| | GGTATCATTGTCGAATTCTCAGCGGAAGACGCGACTCGTACAGAATTAG | |
| | ATTATTTAAAGGAGGTTTATAAGAAGGCCGTCGAAGCTGGAGCTGACAG | |
| | AATCAACGTCCCCGATACTGTCGGTGTTATGGTGCCCCATTCGATGACG | |
| | TATTTGATCTCAGAATTGAAGAAGGACATCAAAGTTCCATTAAGTGTCC | |
| | ACTGTCATAACGACTTTGGTATTGCAGTTTCTAATTCGGTTGCCGCAGT | |
| | TGAAGCAGGTGCTGAACAGGTCCACTGTACAGTCAACGGATTGGGTGAG | |
| | AGAGCAGGCAATGCATCTCTCGAGGAAACCGTGATGACTTTGAATATGG | |
| | TCTATGGTATTGAGACAAACGTTGATACCAAGATGCTAACTAAGCTATC | |
| | TAGAATCGTGTCTAACTACACAGGTATTAAGACACAGCCAAACAAAGCA | |
| | ATTGTCGGTGAGAATTCTTTCGCACACGAATCTGGTATTCATGCGCACG | |
| | GCGTTTTGGCCCACGCATTGACCTACGAGCCGATCGATCCTGCAATCGT | |
| | GGGAAACAAAAGACGTATCGTCTTGGGTAAGCACTCCGGTGCCCACGCG | |
| | ATTAAATCTAAATTGTCTGAGATCGGTGTTGAAATCGGTGACGCTCTAT | |
| | CGAAGGAACAATTCTGTGAAATTGTTGAGAGAGTTAAGGCAATTGGTGA | |
| | TAAGGGTAAGTTGGTTACCGATGCAGATGTCATGGCAATAACAGAGGAT | |
| | ATCACTCAACGAACAATCAAGAGTGAAAGAATTGTCGATTTAGAACAAT | |
| | TCGCAGTCATGACAGGAAATAACGTTCTCCCAACTGCTAGTGTTGCGTT | |
| | GAAGGTCAGAGACAAGATTTATAAAACTTCCGAGTTGGGTGTCGGACCA | |
| | GTTGACGCTGCACTTAAGGCAATCCAGGCGGCGGTGGGAGAAAACATCA | |
| | GGCTCAATGAGTACAATATTTCAGCGATCTCAGGTGGTACTGATGCCAT | |
| | TGCAGAAGTGACAGTTAGATTGGAGAACCATGAGAAGGAAGTCATTGCA | |
| | AAGGCTACTGGTGACGACGTCGTGAAGGCTTCTGTTGAAGCGGTCATAG | |
| | ATGGTATTAATAAACTCATGTCCTAG (SEQ ID NO: 6) | |
| 07* | ATGTCGTTAGTCAAGCTTTATGATACGACTTTGAGAGACGGAACACAGG | From |
| | CAGAGGATATCTCCTTCTTGGTTGAAGATAAGATCAGAATTGCTCATAA | Geobacter |
| | ACTCGATGAGATTGGAATCCACTACATCGAAGGGGGTTGGCCAGGCTCG | sulfurreducens |
| | AACCCCAAGGACGTCGCATTTTTCAAGGATATTAAGAAAGAAAAGTTGT | |
| | CCCAAGCAAAGATCGCGGCGTTTGGTTCCACTAGAAGAGCCAAGGTTAC | |
| | ACCCGATAAGGACCATAACCTCAAAACTCTCATTCAAGCCGAGCCAGAC | |
| | GTCTGTACTATTTTCGGCAAGACATGGGACTTCCACGTCCATGAAGCAT | |
| | TGAGAATTTCCTTGGAAGAAAACTTGGAGCTGATTTTTGATTCGTTGGA | |
| | ATATTTGAAGGCGAACGTACCAGAAGTCTTCTACGATGCAGAACATTTT | |
| | TTTGACGGTTATAAAGCAAATCCAGACTATGCGATTAAGACATTGAAGG | |
| | CAGCTCAGGATGCTAAGGCTGATTGTATTGTCTTGTGTGATACTAACGG | |
| | CGGTACCATGCCATTCGAATTAGTTGAAATTATTAGAGAGGTTAGAAAG | |
| | CATATAACAGCTCCATTGGGTATTCATACTCATAACGACTCCGAATGCG | |
| | CAGTCGCTAACTCTTTACATGCGGTTTCAGAAGGCATTGTGCAAGTTCA | |
| | AGGAACCATCAACGGGTTTGGGGAAAGATGTGGTAACGCAAACTTGTGT | |
| | TCTATTATTCCTGCGTTGAAACTTAAAATGAAAAGAGAATGTATCGGTG | |
| | ATGATCAATTGAGAAAATTGAGAGACTTATCTCGTTTCGTTTACGAGTT | |
| | AGCCAATTTGAGCCCAAACAAACATCAAGCATACGTGGGTAATTCCGCT | |
| | TTCGCCCACAAAGGTGGTGTTCACGTTAGTGCAATCCAACGTCATCCAG | |
| | AAACTTATGAGCACTTGAGACCTGAGTTAGTTGGCAATATGACCAGAGT | |
| | TTTGGTTTCTGACCTATCAGGTAGAAGTAACATTTTGGCAAAAGCGGAA | |
| | GAGTTTAACATCAAAATGGATTCAAAGGACCCAGTCACATTGGAAATCT | |
| | TGGAAAATATTAAGGAAATGGAGAACAGGGGTTACCAATTCGAAGGTGC | |
| | TGAGGCGTCGTTCGAACTGCTGATGAAAAGAGCATTGGGTACTCATCGT | |
| | AAGTTTTTTTCCGTCATTGGTTTTAGAGTTATCGACGAAAAGAGACACG | |
| | AGGATCAAAAGCCATTGTCGGAAGCAACCATCATGGTTAAAGTTGGTGG | |
| | TAAAATTGAACACACAGCAGCTGAAGGTAATGGTCCTGTCAATGCCCTC | |
| | GATAATGCTTTAAGAAAGGCATTAGAGAAGTTCTATCCAAGATTGAAGG | |
| | AAGTTAAACTCTTGGATTATAAGGTTCGTGTCTTACCAGCAGGTCAAGG | |
| | TACCGCCAGTTCTATTCGTGTCCTAATCGAATCGGGTGATAAGGAATCC | |
| | CGTTGGGGTACGGTTGGTGTTTCTGAAAACATAGTGGACGCATCTTACC | |
| | AAGCCCTTCTCGACTCCGTTGAATATAAGCTACATAAGTCGGAAGAAAT | |
| | CGAAGGTTCTAAAAAGTAG (SEQ ID NO: 7) | |
| 08* | ATGACTGCAACGTCAGAACTCGACGACTCATTTCATGTTTTCGATACCA | From |
| | CTTTGAGAGATGGTGCGCAGCGTGAAGGTATTAACTTAACCGTTGCCGA | Streptomyces |
| | CAAGCTCGCTATCGCGAGACACCTTGATGATTTTGGTGTTGGATTTATT | coelicolor |
| | GAAGGAGGCTGGCCAGGTGCAAATCCTAGGGACACAGAATTTTTTGCAA | |
| | GAGCAAGACAAGAAATCGATTTCAAACATGCTCAATTGGTTGCCTTCGG | |
| | CTCTACACGTAGAGCAGGTGCAAATGCCGCAGAAGATCATCAAGTCAAG | |
| | GCATTATTGGATTCGGGTGCACAGGTGATTACATTGGTCGCTAAATCAC | |
| | ACGATAGACACGTTGAATTAGCCCTAAGAACTACCTTGGATGAAAATCT | |
| | TGCGATGGTCGCTGACACCGTTTCCCATCTAAAGGCCCAAGGTAGAAGA | |
| | GTTTTCGTGGATTGTGAACATTTCTTCGATGGATATAGGGCGAACCCTG | |
| | AATACGCTAAGTCCGTTGTTAGAACCGCCTCCGAGGCAGGTGCTGATGT | |
| | TGTTGTTCTGTGTGATACTAATGGCGGCATGTTGCCAGCCCAAATTCAG | |
| | GCCGTTGTTGCAACTGTTCTAGCTGATACGGGTGCCAGACTGGGTATTC | |
| | ATGCGCAGGATGATACCGGTTGTGCCGTCGCAAACACATTAGCCGCTGT | |
| | GGATGCTGGTGCTACTCACGTTCAGTGTACTGCTAATGGTTACGGTGAG | |
| | AGAGTCGGTAACGCAAATTTATTCCCAGTCGTCGCGGCTCTAGAGCTAA | |
| | AATACGGGAAGCAGGTGTTGCCAGAAGGCAGGTTACGGGAAATGACGAG | |
| | AATTTCTCATGCCATCGCTGAGGTCGTCAACTTGACTCCATCTACTCAC | |
| | CAACCATACGTTGGTGTGTCTGCCTTTGCACACAAAGCGGGTCTGCACG | |
| | CTTCAGCGATCAAGGTGGATCCAGATCTTTACCAGCACATTGACCCTGA | |
| | ATTAGTTGGTAACACTATGCGTATGTTAGTGTCTGATATGGCGGGTAGA | |
| | GCGTCAATCGAACTTAAGGGTAAGGAATTGGGCATCGATTTGGGTGGAG | |
| | ACAGAGAGCTTGTAGGTAGAGTTGTTGAAAGAGTTAAGGAGCGTGAGTT | |
| | AGCAGGATACACCTACGAAGCAGCGGATGCATCCTTTGAATTATTATTG | |
| | CGTGCTGAAGCCGAGGGTAGACCACTAAAGTACTTCGAAGTTGAATCCT | |
| | GGAGAGCTATCACCGAGGACAGACCAGATGGCTCTCATGCAAACGAAGC | |
| | TACTGTCAAGTTGTGGGCAAAGGGTGAAAGAATTGTTGCAACTGCTGAA | |
| | GGTAACGGTCCTGTTAATGCACTAGATCGTTCCTTAAGAGTCGCATTGG | |
| | AAAAGATTTATCCTGAATTGGCAAAATTGGATTTGGTCGATTACAAGGT | |
| | CAGGATTTTAGAGGGAGTGCATGGTACACAAAGTACAACAAGGGTTCTA | |
| | ATTTCAACTTCCGACGGGACCGGAGAATGGGCAACTGTGGGTGTTGCTG | |
| | AAAATGTCATTGCTGCATCGTGGCAGGCCTTAGAGGATGCATACACCTA | |
| | TGGTTTATTGCGTGCAGGTGTCGCACCAGCGGAGTAG (SEQ ID | |
| | NO: 8) | |
| 09* | ATGAGTACCTCAATCTCTATTTTTGACACAACCTTGAGAGATGGTACTC | From |
| | AAGGTGAGGGTATCTCTCTTACGGCAGAAGATAAGATTAAAATTGCATT | Arabidopsis |
| | GAAGTTGGACGCACTCGGCGTTCATTATATTGAAGGAGGTAACCCTGGT | thaliana |
| | TCCAACTCCAAGGATATCGAATTCTTTAGAAGAGCACGTGAATTGAATT | |
| | TAAGAGCGAAGCTTACTGCTTTCGGTTCTACCCGTCGTAAGAATTCTTT | |
| | ATGCGAGCAAGACGTTAACTTGTTAAACTTAGTCTCTTCAGGTGCTAAG | |
| | GCGGCAACTCTGGTCGGTAAGACGTGGGACTTCCACGTTCACACAGCTT | |
| | TACAAACTACTTTAGAGGAAAATTTGGCAATGATCTACGATAGCTTAGC | |
| | GTATTTGAAGCAACACGGTTTAGAAGCTATTTTCGACTCCGAGCATTTC | |
| | TTTGATGGCTTCAAGGCTAACCCTGATTATGCTATAGCAGCCTTGAGAA | |
| | AGGCTCAAGAAGCTGGTGCGGACTGGATTGTTTTGTGTGATACAAATGG | |
| | TGGAACCCTTCCAAACGAAATTCAAGATATCGTTAAGCAAGTCAGGAAT | |
| | TCGATCCAAGCGCCAATCGGAATCCACACTCACAATGACTGTGAGCTTG | |
| | CGGTGGCTAATACTTTGGCCGCAGTTACCGCGGGTGCACGTCAGATTCA | |
| | GGGAACTATTAATGGTTACGGTGAAAGATGTGGCAACGCCAATCTATGC | |
| | TCGATTTTACCAACCTTACAGTTGAAGATGGGTTATCAAGTTGTCACTC | |
| | CAGAGCAATTAGGTTCTTTGACATCCGTTGCAAGATACGTTGGTGAAAT | |
| | CGCCAATGTGGTTTTACCTGTGAACCAACCTTATGTTGGTACCGCAGCT | |
| | TTTGCTCACAAAGGTGGTATTCATGTTAGTGCGATTTTGAAGGATTCCA | |
| | AGACATACGAACATATCTCTCCAGATCTGGTCGGTAATAAACAACGTGT | |
| | TTTGGTTTCAGAATTAGCCGGTCAATCGAATATTTTGTCTAAAGCACAG | |
| | GAGATGGGTTTAGCTGTTTCGAACGATAACGCTAACAGTAGAGAAGTTA | |
| | TTGAGAAGATTAAGAACCTGGAGCACCAGGGATATCAATTCGAAGGTGC | |
| | AGATGCATCTTTGGAATTATTGTTGCGTGACGCCTATGGTGATGCAGTT | |
| | GAGATTTTTACTGTTGAAAGCTTTAAGATCTTGATGGAAAAGTCCCCAT | |
| | CTGGTAATTTAACAGAAGCTATTGTCAAATTGAACGTTTCTGGACAACA | |
| | AGTCTACACAGTCGCTGAGGGTAATGGTCCAGTGAACGCTTTAGATAAT | |
| | GCTTTGAGAAAGGCTTTGACCCCCTTTTATCCAGATATCAACGGTATCC | |
| | ATTTGTCCGATTACAAGGTTCGTGTTTTAGACGAAAAAGATACAACGGC | |
| | CGCGAAGGTTAGGGTTCTAATCGAATCTACTAACTTTAAGGAATCTTGG | |
| | TCTACCGTCGGCGTTTCATCTAACGTTATCGAAGCCTCCTGGGAAGCAC | |
| | TTATTGATTCTATTCGTTACGCCTTACTAGGCATGACGCAAACATCCTT | |
| | TTCCCCAGAAAGCCCTAGAGAAAGTTTGGGTTTGGTTAACCATTAA | |
| | (SEQ ID NO: 9) | |
| 10 | ATGGATAATACTGAGACAATTACAGGTGTCGTTAAGCTTCCTGATAGAA | From |
| | CACCAACGAAACACGACATTGCCACTGGTAGAGATCCAGATAGAGTGAA | Chondrus |
| | GATTTTCGACACCACATTGAGGGACGGTGAACAATCACCAGGAGCATCG | crispus |
| | TTGACAGCGGACGAAAAGATGGTTATCGCAAGACAACTCGCTAAGTTGG | |
| | GAGTGGACGTTATTGAGGCTGGGTTCCCAATCGCTTCCGAGGGCGATTT | |
| | TACTGCTGTCAGAGAAATTGCAAAGTCCGTTGGCAACCGTGACAACCCA | |
| | CCAATCATTTGTGGCTTGGCCAGAGCTCTCGAAAAGGATATCTCAAGAT | |
| | GTTACGAAGCCGTCAAGCACGCAGCATTTCCAAGAATCCACACCTTCAT | |
| | CGCAACATCAGATTTGCACATGGCGTATAAATTAAAGAAGACCAGAGAG | |
| | GAAGTCGTTGAAATTACAAAGGAAACAGTTACATATGCCAGAAGTTTGT | |
| | GTGAGGACGTTGAGTTTTCTGCCGAAGATGCCATCAGATCCGATCCAGA | |
| | CTTCCTCTGTGAGGTCTTCTCCGCGGCTATTGAGGCTGGCGCAACTACT | |
| | ATCAACGTTCCTGACACCGTTGGTTACACCACCCCATCGGAATTTGCGT | |
| | CCCTTATTAGATATTTGAGGAGAAACGTCAGAGGTATTGATGACGTTAC | |
| | TATCTCTGTCCACGGACACGATGATTTGGGTATGGCAGTTGCCAACTTC | |
| | TTGTCAGCCGTTGAAAATGGCGCCCGTCAAATGGAGTGTACAATCAACG | |
| | GAATCGGTGAAAGAGCAGGCAACGCTTCTCTGGAAGAAGTCGTGATGGC | |
| | ATTACACGTTAGAAGACAATTCTACAACGCTCGTATGGGAAAGGACAAT | |
| | AAGGTGGACGCGCCGCTAACTAATATAGTTCACAAGGAGATACATCACA | |
| | CCTCTAGAATGGTCAGTAATTTAACTGGAATGCTCGTTCAGCCGAATAA | |
| | GGCAATTGTTGGAGCAAATGCTTTTGCACACGAATCGGGTATCCATCAA | |
| | GACGGTGTTTTAAAGCATAGACAAACGTACGAGATCATGGATGCACAAT | |
| | CCATTGGTTTGTCTGAGAACTCCATTGTCTTGGGTAAGCACTCAGGTAG | |
| | ACACGCATTCAGAACAAGATTGGTTAACATGGGATACGAGGTCACGGAT | |
| | GAAGAGTTGGAAAGAGCATTTAGAAGATTCAAGGAGTTAGCTGACATTA | |
| | AAAAGGAAGTGTCGGAGGCTGATTTACAATCCTTGGTTAACGATGAGGT | |
| | GCGTTTGGTTAAAGAAGCAGTTAAGCTAACTAGAATTCAAATTCAATGT | |
| | GGATACCACATTATTCCAACTGCCACAATCGGTTTGATTTTGGTTGATG | |
| | ATGACGCGGAAAAGACTGTTACATCAACGGGTACTGGTCCTGTTGATTC | |
| | TGCCTACAATGCTATCAACCAAGTCATTGAAGATATCATCCATGTTACT | |
| | CTGCTCGAATACAAGGTTTCATCTGTCTCAAAGGGTATTGATGCGTTAG | |
| | GAGAAGTTGCAGTCCGCGTTCAAGACGGTGCTACTGGTAACCAGTATAT | |
| | CGGTGCGGCAGCCAATACGGATATTGTTGTGGCCTCCGTTCAAGCATAT | |
| | GTTAATGCGATTAACAGATGTCAACTCAATAATAAGAAGCCTAAGATCC | |
| | ATCCACAGTATGGCAACGCGAATTCTGTTTGA (SEQ ID NO: 10) | |

*These genes showed citramalate synthase activity in I. orientalis SD108.

TABLE 5
DNA sequence of I. orientalis-compatible cimA with “mostly used” codon
usage.

| ID | Sequence | Description |
|---|---|---|
| 02 | ATGCAAGTTAAGATTTTGGATACTACTTTGAGAGATGGTGAACA | From |
| | AACTCCAGGTGTTTCTTTGTCTGTTGAACAAAAGGTTATGATTG | Archaeoglobus |
| | CAGAAGCATTGGATAACTTGGGTGTTGATATTATTGAAGCAGGT | fulgidus |
| | ACTGCAATTGCATCTGAAGGTGATTTCCAAGCAATTAAGGAAAT | |
| | TTCTCAAAGAGGTTTGAACGCAGAAATTTGTTCTTTCGCAAGAA | |
| | TTAAGAGAGAAGATATTGATGCAGCAGCAGATGCAGGTGCAGAA | |
| | TCTATTTTCATGGTTGCACCATCTTCTGATATTCATATTAACGC | |
| | AAAGTTCCCAGGTAAGGATAGAGATTACGTTATTGAAAAGTCTG | |
| | TTGAAGCAATTGAATACGCAAAGGAAAGAGGTTTGATTGTTGAA | |
| | TTCGGTGCAGAAGATGCATCTAGAGCAGATTTGGATTTCGTTAT | |
| | TCAATTGTTCAAGAGAGCAGAAGAAGCAAAGGCAGATAGAATTA | |
| | CTTTCGCAGATACTGTTGGTGTTTTGTCTCCAGAAAAGATGGAA | |
| | GAAATTGTTAGAAAGATTAAGGCAAAGGTTAAGTTGCCATTGGC | |
| | AATTCATTGTCATGATGATTTCGGTTTGGCAACTGCAAACACTA | |
| | TTTTCGGTATTAAGGCAGGTGCAGAAGAATTCCATGGTACTATT | |
| | AACGGTTTGGGTGAAAGAGCAGGTAACGCAGCAATTGAAGAAGT | |
| | TGTTATTGCATTGGAATACTTGTACGGTATTAAGACTAAGATTA | |
| | AGAAGGAAAGATTGTACAACACTTCTAAGTTGGTTGAAAAGTTG | |
| | TCTAGAGTTGTTGTTCCACCAAACAAGCCAATTGTTGGTGATAA | |
| | CGCATTCACTCATGAATCTGGTATTCATACTTCTGCATTGTTCA | |
| | GAGATGCAAAGTCTTACGAACCAATTTCTCCAGAAGTTGTTGGT | |
| | AGAAAGAGAGTTATTGTTTTGGGTAAGCATGCAGGTAGAGCATC | |
| | TGTTGAAGCAATTATGAACGAATTGGGTTACAAGGCAACTCCAG | |
| | AACAAATGAAGGAAATTTTGGCAAGAATTAAGGAAATTGGTGAT | |
| | AAGGGTAAGAGAGTTACTGATGCAGATGTTAGAACTATTATTGA | |
| | AACTGTTTTGCAAATTAAGAGAGAAAAGAAGGTTAAGTTGGAAG | |
| | ATTTGGCAATTTTCTCTGGTAAGAACGTTATGCCAATGGCATCT | |
| | GTTAAGTTGAAGATTGATGGTCAAGAAAGAATTGAAGCAGCAGT | |
| | TGGTTTGGGTCCAGTTGATGCAGCAATTAACGCAATTAGAAGAG | |
| | CAATTAAGGAATTCGCAGATATTAAGTTGGTTTCTTACCATGTT | |
| | GATGCAATTACTGGTGGTACTGATGCATTGGTTGATGTTGTTGT | |
| | TCAATTGAAGAAGGATAACAAGATTGTTACTGCAAGAGGTGCAA | |
| | GAACTGATATTATTATGGCATCTGTTGAAGCATTCATTGAAGGT | |
| | ATTAACATGTTGTTCTAA (SEQ ID NO: 11) | |
| 03 | ATGATGGTTAGAATTTTCGATACTACTTTGAGAGATGGTGAACA | From |
| | AACTCCAGGTGTTTCTTTGACTCCAAACGATAAGTTGGAAATTG | Methanocaldococcus |
| | CAAAGAAGTTGGATGAATTGGGTGTTGATGTTATTGAAGCAGGT | jannaschii |
| | TCTGCAATTACTTCTAAGGGTGAAAGAGAAGGTATTAAGTTGAT | |
| | TACTAAGGAAGGTTTGAACGCAGAAATTTGTTCTTTCGTTAGAG | |
| | CATTGCCAGTTGATATTGATGCAGCATTGGAATGTGATGTTGAT | |
| | TCTGTTCATTTGGTTGTTCCAACTTCTCCAATTCATATGAAGTA | |
| | CAAGTTGAGAAAGACTGAAGATGAAGTTTTGGAAACTGCATTGA | |
| | AGGCAGTTGAATACGCAAAGGAACATGGTTTGATTGTTGAATTG | |
| | TCTGCAGAAGATGCAACTAGATCTGATGTTAACTTCTTGATTAA | |
| | GTTGTTCAACGAAGGTGAAAAGGTTGGTGCAGATAGAGTTTGTG | |
| | TTTGTGATACTGTTGGTGTTTTGACTCCACAAAAGTCTCAAGAA | |
| | TTGTTCAAGAAGATTACTGAAAACGTTAACTTGCCAGTTTCTGT | |
| | TCATTGTCATAACGATTTCGGTATGGCAACTGCAAACACTTGTT | |
| | CTGCAGTTTTGGGTGGTGCAGTTCAATGTCATGTTACTGTTAAC | |
| | GGTATTGGTGAAAGAGCAGGTAACGCATCTTTGGAAGAAGTTGT | |
| | TGCAGCATTGAAGATTTTGTACGGTTACGATACTAAGATTAAGA | |
| | TGGAAAAGTTGTACGAAGTTTCTAGAATTGTTTCTAGATTGATG | |
| | AAGTTGCCAGTTCCACCAAACAAGGCAATTGTTGGTGATAACGC | |
| | ATTCGCACATGAAGCAGGTATTCATGTTGATGGTTTGATTAAGA | |
| | ACACTGAAACTTACGAACCAATTAAGCCAGAAATGGTTGGTAAC | |
| | AGAAGAAGAATTATTTTGGGTAAGCATTCTGGTAGAAAGGCATT | |
| | GAAGTACAAGTTGGATTTGATGGGTATTAACGTTTCTGATGAAC | |
| | AATTGAACAAGATTTACGAAAGAGTTAAGGAATTCGGTGATTTG | |
| | GGTAAGTACATTTCTGATGCAGATTTGTTGGCAATTGTTAGAGA | |
| | AGTTACTGGTAAGTTGGTTGAAGAAAAGATTAAGTTGGATGAAT | |
| | TGACTGTTGTTTCTGGTAACAAGATTACTCCAATTGCATCTGTT | |
| | AAGTTGCATTACAAGGGTGAAGATATTACTTTGATTGAAACTGC | |
| | ATACGGTGTTGGTCCAGTTGATGCAGCAATTAACGCAGTTAGAA | |
| | AGGCAATTTCTGGTGTTGCAGATATTAAGTTGGTTGAATACAGA | |
| | GTTGAAGCAATTGGTGGTGGTACTGATGCATTGATTGAAGTTGT | |
| | TGTTAAGTTGAGAAAGGGTACTGAAATTGTTGAAGTTAGAAAGT | |
| | CTGATGCAGATATTATTAGAGCATCTGTTGATGCAGTTATGGAA | |
| | GGTATTAACATGTTGTTGAACTAA (SEQ ID NO: 12) | |
| 07 | ATGTCTTTGGTTAAGTTGTACGATACTACTTTGAGAGATGGTAC | From Geobacter |
| | TCAAGCAGAAGATATTTCTTTCTTGGTTGAAGATAAGATTAGAA | sulfurreducens |
| | TTGCACATAAGTTGGATGAAATTGGTATTCATTACATTGAAGGT | |
| | GGTTGGCCAGGTTCTAACCCAAAGGATGTTGCATTCTTCAAGGA | |
| | TATTAAGAAGGAAAAGTTGTCTCAAGCAAAGATTGCAGCATTCG | |
| | GTTCTACTAGAAGAGCAAAGGTTACTCCAGATAAGGATCATAAC | |
| | TTGAAGACTTTGATTCAAGCAGAACCAGATGTTTGTACTATTTT | |
| | CGGTAAGACTTGGGATTTCCATGTTCATGAAGCATTGAGAATTT | |
| | CTTTGGAAGAAAACTTGGAATTGATTTTCGATTCTTTGGAATAC | |
| | TTGAAGGCAAACGTTCCAGAAGTTTTCTACGATGCAGAACATTT | |
| | CTTCGATGGTTACAAGGCAAACCCAGATTACGCAATTAAGACTT | |
| | TGAAGGCAGCACAAGATGCAAAGGCAGATTGTATTGTTTTGTGT | |
| | GATACTAACGGTGGTACTATGCCATTCGAATTGGTTGAAATTAT | |
| | TAGAGAAGTTAGAAAGCATATTACTGCACCATTGGGTATTCATA | |
| | CTCATAACGATTCTGAATGTGCAGTTGCAAACTCTTTGCATGCA | |
| | GTTTCTGAAGGTATTGTTCAAGTTCAAGGTACTATTAACGGTTT | |
| | CGGTGAAAGATGTGGTAACGCAAACTTGTGTTCTATTATTCCAG | |
| | CATTGAAGTTGAAGATGAAGAGAGAATGTATTGGTGATGATCAA | |
| | TTGAGAAAGTTGAGAGATTTGTCTAGATTCGTTTACGAATTGGC | |
| | AAACTTGTCTCCAAACAAGCATCAAGCATACGTTGGTAACTCTG | |
| | CATTCGCACATAAGGGTGGTGTTCATGTTTCTGCAATTCAAAGA | |
| | CATCCAGAAACTTACGAACATTTGAGACCAGAATTGGTTGGTAA | |
| | CATGACTAGAGTTTTGGTTTCTGATTTGTCTGGTAGATCTAACA | |
| | TTTTGGCAAAGGCAGAAGAATTCAACATTAAGATGGATTCTAAG | |
| | GATCCAGTTACTTTGGAAATTTTGGAAAACATTAAGGAAATGGA | |
| | AAACAGAGGTTACCAATTCGAAGGTGCAGAAGCATCTTTCGAAT | |
| | TGTTGATGAAGAGAGCATTGGGTACTCATAGAAAGTTCTTCTCT | |
| | GTTATTGGTTTCAGAGTTATTGATGAAAAGAGACATGAAGATCA | |
| | AAAGCCATTGTCTGAAGCAACTATTATGGTTAAGGTTGGTGGTA | |
| | AGATTGAACATACTGCAGCAGAAGGTAACGGTCCAGTTAACGCA | |
| | TTGGATAACGCATTGAGAAAGGCATTGGAAAAGTTCTACCCAAG | |
| | ATTGAAGGAAGTTAAGTTGTTGGATTACAAGGTTAGAGTTTTGC | |
| | CAGCAGGTCAAGGTACTGCATCTTCTATTAGAGTTTTGATTGAA | |
| | TCTGGTGATAAGGAATCTAGATGGGGTACTGTTGGTGTTTCTGA | |
| | AAACATTGTTGATGCATCTTACCAAGCATTGTTGGATTCTGTTG | |
| | AATACAAGTTGCATAAGTCTGAAGAAATTGAAGGTTCTAAGAAG | |
| | TAA (SEQ ID NO: 13) | |
| 08 | ATGACTGCAACTTCTGAATTGGATGATTCTTTCCATGTTTTCGA | From Streptomyces |
| | TACTACTTTGAGAGATGGTGCACAAAGAGAAGGTATTAACTTGA | coelicolor |
| | CTGTTGCAGATAAGTTGGCAATTGCAAGACATTTGGATGATTTC | |
| | GGTGTTGGTTTCATTGAAGGTGGTTGGCCAGGTGCAAACCCAAG | |
| | AGATACTGAATTCTTCGCAAGAGCAAGACAAGAAATTGATTTCA | |
| | AGCATGCACAATTGGTTGCATTCGGTTCTACTAGAAGAGCAGGT | |
| | GCAAACGCAGCAGAAGATCATCAAGTTAAGGCATTGTTGGATTC | |
| | TGGTGCACAAGTTATTACTTTGGTTGCAAAGTCTCATGATAGAC | |
| | ATGTTGAATTGGCATTGAGAACTACTTTGGATGAAAACTTGGCA | |
| | ATGGTTGCAGATACTGTTTCTCATTTGAAGGCACAAGGTAGAAG | |
| | AGTTTTCGTTGATTGTGAACATTTCTTCGATGGTTACAGAGCAA | |
| | ACCCAGAATACGCAAAGTCTGTTGTTAGAACTGCATCTGAAGCA | |
| | GGTGCAGATGTTGTTGTTTTGTGTGATACTAACGGTGGTATGTT | |
| | GCCAGCACAAATTCAAGCAGTTGTTGCAACTGTTTTGGCAGATA | |
| | CTGGTGCAAGATTGGGTATTCATGCACAAGATGATACTGGTTGT | |
| | GCAGTTGCAAACACTTTGGCAGCAGTTGATGCAGGTGCAACTCA | |
| | TGTTCAATGTACTGCAAACGGTTACGGTGAAAGAGTTGGTAACG | |
| | CAAACTTGTTCCCAGTTGTTGCAGCATTGGAATTGAAGTACGGT | |
| | AAGCAAGTTTTGCCAGAAGGTAGATTGAGAGAAATGACTAGAAT | |
| | TTCTCATGCAATTGCAGAAGTTGTTAACTTGACTCCATCTACTC | |
| | ATCAACCATACGTTGGTGTTTCTGCATTCGCACATAAGGCAGGT | |
| | TTGCATGCATCTGCAATTAAGGTTGATCCAGATTTGTACCAACA | |
| | TATTGATCCAGAATTGGTTGGTAACACTATGAGAATGTTGGTTT | |
| | CTGATATGGCAGGTAGAGCATCTATTGAATTGAAGGGTAAGGAA | |
| | TTGGGTATTGATTTGGGTGGTGATAGAGAATTGGTTGGTAGAGT | |
| | TGTTGAAAGAGTTAAGGAAAGAGAATTGGCAGGTTACACTTACG | |
| | AAGCAGCAGATGCATCTTTCGAATTGTTGTTGAGAGCAGAAGCA | |
| | GAAGGTAGACCATTGAAGTACTTCGAAGTTGAATCTTGGAGAGC | |
| | AATTACTGAAGATAGACCAGATGGTTCTCATGCAAACGAAGCAA | |
| | CTGTTAAGTTGTGGGCAAAGGGTGAAAGAATTGTTGCAACTGCA | |
| | GAAGGTAACGGTCCAGTTAACGCATTGGATAGATCTTTGAGAGT | |
| | TGCATTGGAAAAGATTTACCCAGAATTGGCAAAGTTGGATTTGG | |
| | TTGATTACAAGGTTAGAATTTTGGAAGGTGTTCATGGTACTCAA | |
| | TCTACTACTAGAGTTTTGATTTCTACTTCTGATGGTACTGGTGA | |
| | ATGGGCAACTGTTGGTGTTGCAGAAAACGTTATTGCAGCATCTT | |
| | GGCAAGCATTGGAAGATGCATACACTTACGGTTTGTTGAGAGCA | |
| | GGTGTTGCACCAGCAGAATAA (SEQ ID NO: 14) | |
| 09 | ATGTCTACTTCTATTTCTATTTTCGATACTACTTTGAGAGATGG | From Arabidopsis |
| | TACTCAAGGTGAAGGTATTTCTTTGACTGCAGAAGATAAGATTA | thaliana |
| | AGATTGCATTGAAGTTGGATGCATTGGGTGTTCATTACATTGAA | |
| | GGTGGTAACCCAGGTTCTAACTCTAAGGATATTGAATTCTTCAG | |
| | AAGAGCAAGAGAATTGAACTTGAGAGCAAAGTTGACTGCATTCG | |
| | GTTCTACTAGAAGAAAGAACTCTTTGTGTGAACAAGATGTTAAC | |
| | TTGTTGAACTTGGTTTCTTCTGGTGCAAAGGCAGCAACTTTGGT | |
| | TGGTAAGACTTGGGATTTCCATGTTCATACTGCATTGCAAACTA | |
| | CTTTGGAAGAAAACTTGGCAATGATTTACGATTCTTTGGCATAC | |
| | TTGAAGCAACATGGTTTGGAAGCAATTTTCGATTCTGAACATTT | |
| | CTTCGATGGTTTCAAGGCAAACCCAGATTACGCAATTGCAGCAT | |
| | TGAGAAAGGCACAAGAAGCAGGTGCAGATTGGATTGTTTTGTGT | |
| | GATACTAACGGTGGTACTTTGCCAAACGAAATTCAAGATATTGT | |
| | TAAGCAAGTTAGAAACTCTATTCAAGCACCAATTGGTATTCATA | |
| | CTCATAACGATTGTGAATTGGCAGTTGCAAACACTTTGGCAGCA | |
| | GTTACTGCAGGTGCAAGACAAATTCAAGGTACTATTAACGGTTA | |
| | CGGTGAAAGATGTGGTAACGCAAACTTGTGTTCTATTTTGCCAA | |
| | CTTTGCAATTGAAGATGGGTTACCAAGTTGTTACTCCAGAACAA | |
| | TTGGGTTCTTTGACTTCTGTTGCAAGATACGTTGGTGAAATTGC | |
| | AAACGTTGTTTTGCCAGTTAACCAACCATACGTTGGTACTGCAG | |
| | CATTCGCACATAAGGGTGGTATTCATGTTTCTGCAATTTTGAAG | |
| | GATTCTAAGACTTACGAACATATTTCTCCAGATTTGGTTGGTAA | |
| | CAAGCAAAGAGTTTTGGTTTCTGAATTGGCAGGTCAATCTAACA | |
| | TTTTGTCTAAGGCACAAGAAATGGGTTTGGCAGTTTCTAACGAT | |
| | AACGCAAACTCTAGAGAAGTTATTGAAAAGATTAAGAACTTGGA | |
| | ACATCAAGGTTACCAATTCGAAGGTGCAGATGCATCTTTGGAAT | |
| | TGTTGTTGAGAGATGCATACGGTGATGCAGTTGAAATTTTCACT | |
| | GTTGAATCTTTCAAGATTTTGATGGAAAAGTCTCCATCTGGTAA | |
| | CTTGACTGAAGCAATTGTTAAGTTGAACGTTTCTGGTCAACAAG | |
| | TTTACACTGTTGCAGAAGGTAACGGTCCAGTTAACGCATTGGAT | |
| | AACGCATTGAGAAAGGCATTGACTCCATTCTACCCAGATATTAA | |
| | CGGTATTCATTTGTCTGATTACAAGGTTAGAGTTTTGGATGAAA | |
| | AGGATACTACTGCAGCAAAGGTTAGAGTTTTGATTGAATCTACT | |
| | AACTTCAAGGAATCTTGGTCTACTGTTGGTGTTTCTTCTAACGT | |
| | TATTGAAGCATCTTGGGAAGCATTGATTGATTCTATTAGATACG | |
| | CATTGTTGGGTATGACTCAAACTTCTTTCTCTCCAGAATCTCCA | |
| | AGAGAATCTTTGGGTTTGGTTAACCATTAA (SEQ ID | |
| | NO: 15) | |

TABLE 6
PacBio sequencing statistics.

| | All Contigs | | | |
|---|---|---|---|---|
| Statistics | SB814 | SB815 | SB816 | SB817 |
| Number of | 1,191 | 1,225 | 1,016 | 1,412 |
| contigs | | | | |
| Min Length (bp) | 3,834 | 4,544 | 3,504 | 3,134 |
| Max Length (bp) | 792,938 | 825,555 | 1,074,452 | 731,805 |
| Mean Length (bp) | 118,186 | 121,428 | 109,390 | 110,307 |
| N50 Length (bp) | 190,901 | 193,702 | 171,200 | 177,179 |
| Number of | 216 | 242 | 188 | 269 |
| contigs ≥N50 | | | | |
| Length Sum | 140,760,700 | 148,749,713 | 111,140,318 | 155,753,959 |
| Avg coverage | 12.8 | 13.5 | 10.1 | 14.2 |

The sequencing statistics were generated by Geneious Prime version 2022.2.2.

TABLE 7
Protein sequence % identity of CimA
variants with M. jannaschii CimA.

| CimA variants | % identity with M. jannaschii CimA* |
|---|---|
| A. fulgidus (gene #02), | 53.56 |
| G. sulfurreducens (gene #07) | 29.62 |
| S. coelicolor (gene #08) | 31.17 |
| A. thaliana (gene #09) | 32.11 |

*% identity results were generated using NCBI BLAST: Basic Local Alignment Search Tool (Altschul et al., 1990).

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

All cited references are hereby each specifically incorporated by reference in their entireties.

What is claimed is:

1. A genetically modified yeast host cell comprising a heterologous citramalate synthase, and knocked out or reduced in expression, or under conditional expression, for an endogenous or native pyruvate decarboxylase (PDC) gene.

2. The genetically modified yeast host cell of claim 1, wherein the yeast host cell is an Issatchenkia cell.

3. The genetically modified yeast host cell of claim 2, wherein the Issatchenkia cell is an Issatchenkia hanoiensis or Issatchenkia orientalis cell.

4. The genetically modified yeast host cell of claim 1, wherein the heterologous citramalate synthase has an amino acid sequence having at least about 70% amino acid sequence identity with any one citramalate synthase encoded by SEQ ID NOs: 1-10.

5. The genetically modified yeast host cell of claim 4, wherein the heterologous citramalate synthase has an amino acid sequence of at least about 80% amino acid sequence identity.

6. The genetically modified yeast host cell of claim 4, wherein the heterologous citramalate synthase has an amino acid sequence of at least about 90% amino acid sequence identity.

7. The genetically modified yeast host cell of claim 4, wherein the heterologous citramalate synthase has an amino acid sequence of at least about 95% amino acid sequence identity.

8. The genetically modified yeast host cell of claim 4, wherein the heterologous citramalate synthase has an amino acid sequence of at least about 99% amino acid sequence identity.

9. A method for constructing a genetically modified yeast host cell capable of producing citramalate, comprising: (a) introducing a nucleic acid encoding a heterologous citramalate synthase operatively linked to a promoter in a yeast host cell, and (b) deleting, knocking out, or reducing the expression for an endogenous or native pyruvate decarboxylase (PDC) gene in the yeast host cell to produce the genetically modified yeast host cell of claim 1.

10. A method for producing citramalate, comprising: (a) introducing the genetically modified yeast host cell of claim 1 to a culture medium, and (b) growing or culturing the genetically modified yeast host such that the genetically modified yeast host produce citramalate.

11. The method of claim 10, comprising (c) separating the citramalate from the genetically modified yeast host cell and/or the culture medium.
